(19) United States
(12) Patent Application Publication
(10) Pub. No.: US 2021/0317403 A1
(43) Pub. Date: Oct. 14, 2021

(54) STEM CELL-DERIVED CELL CULTURES, STEM CELL-DERIVED THREE DIMENSIONAL TISSUE PRODUCTS, AND METHODS OF MAKING AND USING THE SAME

(71) Applicant: The Regents of the University of Colorado, A Body Corporate, Denver, CO (US)

(72) Inventors: Maria VALERIA CANTO-SOLER, Denver, CO (US); Miguel FLORES BELLVER, Centennial, CO (US); Kang LI, Aurora, CO (US); Silvia APARICIO DOMINGO, Centennial, CO (US)

(21) Appl. No.: 17/053,422

(22) PCT Filed: May 9, 2019
(86) PCT No.: PCT/US2019/031442

§ 371 Date: Nov. 6, 2020

Related U.S. Application Data
(60) Provisional application No. 62/826,196, filed on Mar. 29, 2019.
(60) Provisional application No. 62/669,133, filed on May 9, 2018.

(51) Int. Cl. C12N 5/00; C12N 5/079; A61K 35/30; G01N 33/50

(52) U.S. Cl. CPC C12N 5/0062; C12N 5/0621; A61K 35/30; C12N 2502/085; C12N 2513/00; C12N 2527/00; G01N 2800/164; G01N 33/5058

(57) ABSTRACT

Provided herein are methods for generating stem cell-derived retinal pigment epithelial monolayer cultures as well as methods of using the same. Also provided are populations of retinal pigment epithelial cells prepared according to these methods. In addition, three-dimensional tissue products derived from human induced pluripotent stem cells are also provided along with methods of making and using the same.

### RELATED APPLICATIONS

This application is a U.S. National Phase Application, filed under 35 U.S.C. § 371, of International Application No. PCT/US2019/031442, filed May 9, 2019, which claims priority to, and the benefit of, U.S. Provisional Application No. 62/669,133, filed May 9, 2018 and U.S. Provisional Application No. 62/826,196, filed Mar. 29, 2019, the contents of each of which are incorporated herein by reference in their entireties.

### GOVERNMENT SUPPORT

This invention was made with government support under Grant Number R01EY022631 awarded by the National Institutes of Health/National Eye Institute. The government has certain rights in the invention.

### FIELD OF THE INVENTION

The present invention relates generally to the field of stem cells. More specifically, the invention provides methods for generating stem cell-derived retinal pigment epithelial (RPE) monolayer cultures from human retinal organoids, three-dimensional tissue products derived from human induced pluripotent stem cells, and methods of making and using the same.

### BACKGROUND OF THE INVENTION

Retinal degenerative diseases are a group of clinical conditions in which the dysfunction and death of retinal photoreceptor cells lead to irreversible vision loss, and sometimes, total blindness. Currently, there is no treatment available to prevent many retinal degenerative diseases. Thus, there remains a need in the art to develop means to study retinal development, cell interactions, and physiological and disease mechanisms. In addition, there is also a need to develop additional treatments for these diseases.

### SUMMARY OF THE INVENTION

Provided herein are three-dimensional tissue products derived from human induced pluripotent stem cells (hiPSCs) containing functionally matured retinal pigment epithelial (RPE) cells and portion of three-dimensional neural retina (3DNR), wherein the 3DNR and the RPE cells are physically and functionally integrated to form a complex containing a layer of neural retina and an underlying layer of RPE cells. In various embodiments, in this three-dimensional tissue products, the RPE cells and the 3DNR are both obtained from human retinal organoids.

In embodiments, the 3DNR may include: i) undifferentiated pseudostratified neural retinal epithelium; ii) laminated neural retina tissue including all retinal layers and their corresponding retinal precursor cell types; and/or iii) advanced differentiated retinal tissue including an outer nuclear layer (ONL) and a bipolar cell layer (BCL), wherein the ONL could be rod-enriched, cone-enriched, or any combination thereof.

In these products, the RPE cells can be prepared according to any of the methods described herein or according to any methods known in the art that generate similar RPE tissue. In various embodiments, the RPE cells are: i) obtained from the initial plating or any passage thereafter; ii) at early stages of differentiation; and/or iii) at more advanced stages of differentiation, times in culture, or combinations thereof.

Any of the three-dimensional tissue products described herein can also contain an additional biocompatible component integrated into the product. By way of non-limiting example, the additional biocompatible component can be a natural or synthetic compound in a liquid or gel form (e.g., a hydrogel) that provides an appropriate biomechanical environment for cell survival and function and/or allows manipulation of the product. The inclusion of this additional biocompatible component promotes survival and function of the transplanted cells.

Additionally or alternatively, any of the three-dimensional tissue products described herein may further contain a biocompatible scaffold, wherein the RPE cells are grown on top of said scaffold prior to integration with the 3DNR. By way of non-limiting example, such biocompatible scaffolds may include natural or synthetic scaffolds, scaffolds made from biodegradable materials, scaffolds made from or non-biodegradable materials, or any combinations thereof.

Also provided herein are three-dimensional tissue products derived from human induced pluripotent stem cells (hiPSCs) containing functionally matured retinal pigment epithelial (RPE) cells and portion of three-dimensional neural retina (3DNR), wherein the 3DNR and the RPE cells are physically and functionally integrated to form a complex containing a layer of neural retina and an underlying layer of RPE cells. In various embodiments, in this three-dimensional tissue products, the RPE cells and the 3DNR are both obtained from human retinal organoids.

In embodiments, the 3DNR may include: i) undifferentiated pseudostratified neural retinal epithelium; ii) laminated neural retina tissue including all retinal layers and their corresponding retinal precursor cell types; and/or iii) advanced differentiated retinal tissue including an outer nuclear layer (ONL) and a bipolar cell layer (BCL), wherein the ONL could be rod-enriched, cone-enriched, or any combination thereof.

In these products, the RPE cells can be prepared according to any of the methods described herein or according to any methods known in the art that generate similar RPE tissue. In various embodiments, the RPE cells are: i) obtained from the initial plating or any passage thereafter; ii) at early stages of differentiation; and/or iii) at more advanced stages of differentiation, times in culture, or combinations thereof.

Any of the three-dimensional tissue products described herein can also contain an additional biocompatible component integrated into the product. By way of non-limiting example, the additional biocompatible component can be a natural or synthetic compound in a liquid or gel form (e.g., a hydrogel) that provides an appropriate biomechanical environment for cell survival and function and/or allows manipulation of the product. The inclusion of this additional biocompatible component promotes survival and function of the transplanted cells.

Additionally or alternatively, any of the three-dimensional tissue products described herein may further contain a biocompatible scaffold, wherein the RPE cells are grown on top of said scaffold prior to integration with the 3DNR. By way of non-limiting example, such biocompatible scaffolds may include natural or synthetic scaffolds, scaffolds made from biodegradable materials, scaffolds made from or non-biodegradable materials, or any combinations thereof.

In embodiments, three-dimensional tissue products are derived from human induced pluripotent stem cells (hiPSCs) containing functionally matured retinal pigment epithelial (RPE) cells and a portion of three-dimensional neural retina (3DNR), an additional biocompatible component, and a biocompatible scaffold, wherein the 3DNR, the RPE cells, and the additional biocompatible component are physically and functionally integrated to form a complex containing a layer of neural retina and an underlying layer of RPE cells, wherein the 3DNR contains: i) undifferentiated pseudostratified neural retinal epithelium; ii) laminated neural retina tissue including all retinal layers and their corresponding retinal precursor cell types; and/or iii) advanced differentiated retinal tissue including an outer nuclear layer (ONL) and a bipolar cell layer (BCL), wherein the ONL could be rod-enriched, cone-enriched, or any combination thereof, wherein the additional biocompatible component contains a natural or synthetic compound in a liquid or gel form that provides an appropriate biomechanical environment for cell survival and function, allows manipulation of the product, or both, and wherein the RPE cells are grown on top of the same or a different biocompatible scaffold prior to integration with the 3DNR, and wherein the 3DNR is positioned on top of the RPE cells.

In these three-dimensional tissue products, the RPE cells and the 3DNR can both be obtained from human retinal organoids.

Likewise, in these three-dimensional tissue products, the RPE cells can be prepared by: a) culturing human retinal organoids in a first culture medium that is not supplemented with exogenous growth factors, morphogenes, or modulators of their signaling pathways, to generate RPE cells and neural retina (NR); b) isolating RPE tissue from the cultured retinal organoids; c) dissociating the isolated RPE tissue into a suspension of single RPE cells; d) plating single RPE cells in an adherent culture; and/or e) culturing the plated cells in a second culture medium that is not supplemented with exogenous growth factors, morphogenes, or modulators of their signaling pathways, to produce a monolayer of RPE.

In any of these three-dimensional tissue products, the RPE cells can be: i) obtained from the initial plating or any passage thereafter; ii) at early stages of differentiation; and/or iii) at more advanced stages of differentiation, times in culture, or combinations thereof.

Suitable biocompatible scaffolds include, but are not limited to, natural or synthetic scaffolds, scaffolds made from biodegradable materials, scaffolds made from or non-biodegradable materials, or any combinations thereof.

Also provided herein are methods of making the three-dimensional tissue product derived from human induced pluripotent stem cells (hiPSCs) containing functionally matured retinal pigment epithelial (RPE) cells and a neural retinal patch obtained from three-dimensional neural retina (3DNR) by: a) culturing human retinal organoid to generate RPE cells and 3DNR; b) separating the RPE cells and the 3DNR; c) seeding the neural retinal patch on top of the RPE cells to form a complex; and d) co-culturing the complex in a suitable culture medium, wherein, following co-culture, the 3DNR and the RPE cells physically and functionally integrate to form a three-dimensional tissue product containing a layer of neural retina and an underlying layer of RPE cells.

In some embodiments, prior to step c), the RPE cells are cultured to generate an RPE monolayer culture.

In one non-limiting embodiment, the RPE monolayer culture is generated by: i) dissociating RPE cells into a suspension of single RPE cells; ii) plating single RPE cells in an adherent culture; and iii) culturing the plated cells in a second culture medium that is not supplemented with exogenous growth factors, morphogenes, or modulators (i.e., agonists and/or antagonists) to produce a monolayer of RPE

In embodiments, the 3DNR may include: i) undifferentiated pseudostratified neural retinal epithelium; ii) laminated neural retina tissue including all retinal layers and their corresponding retinal precursor cell types; and/or iii) advanced differentiated retinal tissue including an outer nuclear layer (ONL) and a bipolar cell layer (BCL), wherein the ONL could be rod-enriched, cone-enriched, or any combination thereof.

In these products, the RPE cells can be prepared according to any of the methods described herein or according to any methods known in the art that generate similar RPE tissue. In various embodiments, the RPE cells are: i) obtained from the initial plating or any passage thereafter; ii) at early stages of differentiation; and/or iii) at more advanced stages of differentiation, times in culture, or combinations thereof.

In any of these methods, the RPE cells are dissociated into single RPE cells using an enzymatic reaction (e.g., using collagenase, trypsin, dispase, TrypLE, papain, and/or any combinations thereof), an enzyme-free dissociation solution, or mechanical means (e.g., mechanical dissociation).

In various embodiments, the single RPE cells can be plated a density between about 25,000 to about 300,000 cells per cm² (i.e., about 25,000; 50,000; 75,000; 100,000; 125,000; 150,000; 175,000; 200,000; 225,000; 250,000; 275,000; or 300,000 cells per cm². For example, the single RPE cells can be plated at a density of approximately 100,000 cells per cm².

In step e) of this method, the second culture medium can be any culture medium that supports the growth of the RPE cells. By way of non-limiting example, this second culture medium can include one or more of the following components: minimal essential media (MEM) a modification, N1 supplement, glutamine, penicillin, streptomycin, non-essential amino acids, taurine, hydrocortisone, triiodo thyronin, and/or fetal bovine serum. Determination of the appropriate components for the second culture medium is within the routine level of skill in the art.

In these methods, the second culture medium can be changed periodically (e.g., every 1, 2, 3, 4, 5, 6, or more days). Likewise, the cells in the adherent culture can be periodically passaged. For example, the cells can be passaged every 1, 2, 3, 4, 5, 6, 7, 8, 9, 10, 11, 12, 13, 14, 15, 16, 17, 18, 19, 20, or more days. In one non-limiting embodiment, the cells are passaged every 10 days to insure that a full monolayer has developed and that the cells start to develop an irregular cobblestone shape. In other embodiments, the cells within the resulting monolayer culture retain their RPE differentiation and maturation capacity until at least passage 4 (i.e., at least passage 4, 5, 6, 7, 8, 9, 10, or more), without the addition of exogenous factors.

As noted, the human retinal organoids can be prepared by any method(s) known in the art. By way of non-limiting example, the human retinal organoids can be prepared by i) culturing hiPSCs to form aggregates; ii) transitioning the aggregates into neural induction medium; iii) seeding the aggregates onto extracellular matrix coated cell culture substrates; iv) replacing the neural induction medium with a chemically-defined differentiating medium; v) detaching NR domains; vi) culturing in suspension to form three-dimensional retinal organoids; and/or vii) adding animal serum or plasma component and retinoic acid. Routine modifications to this method are within the routine level of skill in the art.

Any of the methods of the three-dimensional tissue products disclosed herein can additionally involve the further step of e) embedding the neural retinal patch from the 3DNR, the RPE cells or both the neural retinal patch from the 3DNR and the RPE cells in an additional biocompatible component integrated into the product. By way of non-limiting example, the additional biocompatible component is a natural or synthetic compound in a liquid or gel form (e.g., a hydrogel) that provides an appropriate biomechanical environment for cell survival and function, allows manipulation of the product, or both. The inclusion of this additional biocompatible component promotes survival and function of the transplanted cells.

In any of these methods, the RPE monolayer is grown on top of a biocompatible scaffold prior to integration with the 3DNR. Suitable biocompatible scaffolds include, but are not limited to, natural or synthetic scaffolds, scaffolds made from biodegradable materials, scaffolds made from or non-biodegradable materials, or any combinations thereof.

In any of the methods described herein, the 3DNR and the RPE cells can be co-cultured at different times of cell maturation. In some embodiments, the 3DNR and the RPE cells are co-cultured in a culture medium that results in a rod-enriched three-dimensional tissue product. In alternative embodiments, the 3DNR and the RPE cells are co-cultured in a culture medium that results in a cone-enriched three-dimensional tissue product. Those skilled in the art will recognize that any combination of rod-enriched and/or cone-enriched cells may be used in any of the three-dimensional tissue constructs described herein.

In embodiments, the disclosure provides methods of making a three-dimensional tissue product derived from human induced pluripotent stem cells (hiPSCs) containing functionally matured retinal pigment epithelial (RPE) cells and a neural retinal patch obtained from three-dimensional neural retina (3DNR), an additional biocompatible component, and a biocompatible scaffold. For example, these methods may include the steps of a) culturing human retinal organoid to generate RPE cells and 3DNR; b) separating the RPE cells and the 3DNR; c) seeding the neural retinal patch on top of the RPE cells to form a complex; d) co-culturing the complex in a suitable culture medium; and/or e) embedding the neural retinal patch from the 3DNR, the RPE cells or both the neural retinal patch from the 3DNR and the RPE cells in an additional biocompatible component integrated into the product, wherein, following co-culture, the 3DNR, the RPE cells, and the additional biocompatible component physically and functionally integrate to form a three-dimensional tissue product containing a layer of neural retina and an underlying layer of RPE cells, wherein the 3DNR contains: i) undifferentiated pseudostratified neural retinal epithelium; ii) laminated neural retina tissue including all retinal layers and their corresponding retinal precursor cell types; and/or iii) advanced differentiated retinal tissue including an outer nuclear layer (ONL) and a bipolar cell layer (BCL), wherein the ONL could be rod-enriched, cone-enriched, or any combination thereof, wherein the additional biocompatible component includes a natural or synthetic compound in a liquid or gel form that provides an appropriate biomechanical environment for cell survival and function, allows manipulation of the product, or both, and wherein the RPE cells are grown on top of the same or a different biocompatible scaffold prior to integration with the 3DNR, and wherein the 3DNR is positioned on top of the RPE cells.

In any of these methods, prior to step c), the RPE cells can be cultured to generate an RPE monolayer culture. For example, the RPE monolayer culture can be generated by i) dissociating RPE cells into a suspension of single RPE cells; ii) plating single RPE cells in an adherent culture; and/or iii) culturing the plated cells in a second culture medium that is not supplemented with exogenous growth factors, morphogenes, or modulators of their signaling pathways, to produce a monolayer of RPE.

RPE cells used in these methods can be i) obtained from the initial plating or any passage thereafter; ii) at early stages of differentiation; and/or iii) at more advanced stages of differentiation, times in culture, or combinations thereof. Moreover, in any of these methods, the RPE cells can be dissociated into single RPE cells using an enzymatic reaction, an enzyme-free dissociation solution, and/or mechanical means (e.g., the dissociated RPE tissue is mechanically dissociated).

In embodiments, the single RPE cells are plated at a density between about 25,000 and about 300,000 cells per cm², for example, at a density of approximately 100,000 cells per cm².

In any of these methods, the second culture medium supports the growth of the RPE cells.

Suitable biocompatible scaffolds include, but are not limited to, natural or synthetic scaffolds, scaffolds made from biodegradable materials, scaffolds made from non-biodegradable materials, or any combinations thereof.

In embodiments, the 3DNR and the RPE cells are co-cultured at different times of cell maturation; the 3DNR and the RPE cells are co-cultured in a culture medium that results in a rod-enriched three-dimensional tissue product; and/or the 3DNR and the RPE cells are co-cultured in a culture medium that results in a cone-enriched three-dimensional tissue product.

Also provided are methods of treating a retinal disease, disorder, or condition, by transplanting any of the three-dimensional tissue products described herein into an eye of a patient in need thereof. By way of non-limiting example, the retinal disease, disorder, or condition can be selected from the group consisting of retinitis pigmentosa (RP), Leber's congenital amaurosis (LCA), Stargardt disease, Usher's syndrome, choroideremia, a rod-cone or cone-rod dystrophy, a ciliopathy, a mitochondrial disorder, progressive retinal atrophy, a degenerative retinal disease, age related macular degeneration (AMD), wet AMD, dry AMD, geographic atrophy, a familial or acquired maculopathy, a retinal photoreceptor disease, a retinal pigment epithelial-based disease, diabetic retinopathy, cystoid macular edema, uveitis, retinal detachment, traumatic retinal injury, iatrogenic retinal injury, macular holes, macular telangiectasia, a ganglion cell disease, an optic nerve cell disease, glaucoma, optic neuropathy, ischemic retinal disease, retinopathy of prematurity, retinal vascular occlusion, familial macroaneurysm, a retinal vascular disease, an ocular vascular diseases, a vascular disease, and/or ischemic optic neuropathy.

Also provided are any of the three-dimensional tissue products described herein for use in treating a retinal disease, disorder, or condition. The product is for transplantation into the eye of a patient in need thereof. By way of non-limiting example, the retinal disease, disorder, or condition can be selected from the group consisting of retinitis pigmentosa (RP), Leber's congenital amaurosis (LCA), Stargardt disease, Usher's syndrome, choroideremia, a rod-cone or cone-rod dystrophy, a ciliopathy, a mitochondrial disorder, progressive retinal atrophy, a degenerative retinal disease, age related macular degeneration (AMD), wet AMD, dry AMD, geographic atrophy, a familial or acquired maculopathy, a retinal photoreceptor disease, a retinal pigment epithelial-based disease, diabetic retinopathy, cystoid macular edema, uveitis, retinal detachment, traumatic retinal injury, iatrogenic retinal injury, macular holes, macular telangiectasia, a ganglion cell disease, an optic nerve cell disease, glaucoma, optic neuropathy, ischemic retinal disease, retinopathy of prematurity, retinal vascular occlusion, familial macroaneurysm, a retinal vascular disease, an ocular vascular diseases, a vascular disease, and/or ischemic optic neuropathy.

Additionally provided herein are methods of screening for agents that effect retinal development, function, proliferation, maturation, differentiation, survival, or any combination thereof, by a) contacting any of the three-dimensional tissue products described herein with at least one agent (e.g., a biological agent); and b) determining if said agent has an effect on retinal development, function, proliferation, maturation, differentiation, survival, or any combination thereof. For example, the biological agent can be a growth factor, a trophic factor, a regulatory factor, a hormone, an antibody or an antigen-binding fragment thereof, small molecule, and/or a peptide.

Any of the three-dimensional tissue products described herein can be used to examine retinal development. For example, provided herein are in vitro methods for examining retinal development by: a) preparing the three-dimensional tissue product; and b) monitoring the cellular interaction, function, proliferation, maturation, differentiation, survival, or any combination thereof of cells within the three-dimensional tissue product. Such monitoring may provide information regarding normal retinal development (i.e., information regarding the interaction of the retina and the RPE) and/or information regarding retinal abnormal development, diseases, disorders, or conditions (i.e., information regarding underlying mechanisms of retinal abnormal development, diseases, disorders, or conditions).

Also provided herein are methods for generating stem cell-derived retinal pigment epithelial (RPE) monolayer cultures by a) culturing human retinal organoids in a first culture medium that is not supplemented with exogenous growth factors, morphogenes, or modulators (i.e., agonists and/or antagonists) of their signaling pathways, to generate RPE cells and neural retina (NR); b) isolating RPE tissue from the cultured retinal organoids (e.g., by dissecting the RPE cells from the retinal organoid); c) dissociating the isolated RPE tissue into a suspension of single RPE cells (e.g., by dissociating into single RPE cells using an enzymatic reaction (e.g., using collagenase, trypsin, dispase, TrypLE, papain, and/or any combinations thereof), an enzyme-free dissociation solution, mechanical means, or any combinations thereof); d) plating single RPE cells in an adherent culture; and e) culturing the plated cell in a second culture medium that is not supplemented with exogenous growth factors, morphogenes, or modulators (i.e., agonists and/or antagonists), to produce a monolayer of RPE. In various embodiments, the human retinal organoids are three-dimensional retinal organoids that are derived from human induced pluripotent stem cells (hiPSCs).

The human retinal organoids can be prepared by any method(s) known in the art. By way of non-limiting example, the human retinal organoids can be prepared by i) culturing hiPSCs to form aggregates; ii) transitioning the aggregates into neural induction medium; iii) seeding the aggregates onto extracellular matrix coated cell culture substrates; iv) replacing the neural induction medium with a chemically-defined differentiating medium; v) detaching NR domains; vi) culturing in suspension to form three-dimensional retinal organoids; and/or vii) adding animal serum or plasma component and retinoic acid. Routine modifications to this method are within the routine level of skill in the art.

In any of the methods described herein, the RPE cells that are generated are found as a clump of monolayer polarized RPE tissue or a disorganized RPE tissue associated to the retinal organoids. Those skilled in the art will recognize that the RPE tissue can be mechanically dissected from the retinal organoid and/or that the dissociated RPE tissue can be mechanically dissociated.

In various embodiments, the single RPE cells can be plated a density between about 25,000 to about 300,000 cells per cm² (i.e., about 25,000; 50,000; 75,000; 100,000; 125,000; 150,000; 175,000; 200,000; 225,000; 250,000; 275,000; or 300,000 cells per cm²). For example, the single RPE cells can be plated at a density of approximately 100,000 cells per cm².

In step e) of this method, the second culture medium can be any culture medium that supports the growth of the RPE cells. By way of non-limiting example, this second culture medium can include one or more of the following components: minimal essential media (MEM) a modification, N1 supplement, glutamine, penicillin, streptomycin, non-essential amino acids, taurine, hydrocortisone, triiodo thyronin, and/or fetal bovine serum. Determination of the appropriate components for the second culture medium is within the routine level of skill in the art.

In these methods, the second culture medium can be changed periodically (e.g., every 1, 2, 3, 4, 5, 6, or more days). Likewise, the cells in the adherent culture can be periodically passaged. For example, the cells can be passaged every 1, 2, 3, 4, 5, 6, 7, 8, 9, 10, 11, 12, 13, 14, 15, 16, 17, 18, 19, 20, or more days. In one non-limiting embodiment, the cells are passaged every 10 days to insure that a full monolayer has developed and that the cells start to develop a regular cobblestone shape. In other embodiments, the cells within the resulting monolayer culture retain their RPE differentiation and maturation capacity until at least passage 4 (i.e., at least passage 4, 5, 6, 7, 8, 9, 10, or more), without the addition of exogenous factors.

Using any of the methods described herein, the RPE cells in the monolayer express functional, molecular, and/or cellular features of primary RPE cells.

By way of non-limiting example, the RPE cells in the monolayer may express specific molecules associated with differentiation and functional maturation of RPE cells including, but not limited to, vascular endothelial growth factor (VEGF), melanogenesis associated transcription factor (MITF), ezrin, retinal pigment epithelium-specific 65 kDa protein (RPE65); zonula occludens-1 (ZO-1); bestrophin-1 (BEST1); cellular retinaldehyde-binding protein (CRALBP); lecithin retinol acyltransferase (LRAT); tyrosinase (TYR); pigment epithelium-derived factor (PEDF), tryrosinase, premelanosome protein (PMEL), Claudin3, receptor tyrosine k kinase (MERKT), orthodenticle homeobox 2 (OTX2), and combinations thereof.

Additionally (or alternatively), the RPE cells in the monolayer achieve proper polarization with formation of specialized functional structures of RPE including abundant apical microvilli, adherents junctions, tight junctions, transepithelial resistance (TER), or any combinations thereof.

Also provided herein is a population containing retina pigmented epithelial (RPE) cells prepared according to any of the methods described herein. The resulting population can be utilized in any of the methods described herein.

The disclosure also provides methods of treating a retinal disease, disorder, or condition, the method comprising administering an effective amount of RPE cells prepared according to any of the methods described herein to a patient in need thereof. By way of non-limiting example, the retinal disease, disorder, or condition can be selected from retinitis pigmentosa (RP), Leber's congenital amaurosis (LCA), Stargardt disease, Usher's syndrome, choroideremia, a rod-cone or cone-rod dystrophy, a ciliopathy, a mitochondrial disorder, progressive retinal atrophy, a degenerative retinal disease, age related macular degeneration (AMD), wet AMD, dry AMD, geographic atrophy, a familial or acquired maculopathy, a retinal photoreceptor disease, a retinal pigment epithelial-based disease, diabetic retinopathy, cystoid macular edema, uveitis, retinal detachment, traumatic retinal injury, iatrogenic retinal injury, macular holes, macular telangiectasia, a ganglion cell disease, an optic nerve cell disease, glaucoma, optic neuropathy, ischemic retinal disease, retinopathy of prematurity, retinal vascular occlusion, familial macroaneurysm, a retinal vascular disease, an ocular vascular diseases, a vascular disease, and/or ischemic optic neuropathy.

The disclosure also provides RPE cells prepared according to any of the methods described herein for use in treating a retinal disease, disorder, or condition. The RPE cells are for administration in an effective amount in a patient in need thereof. By way of non-limiting example, the retinal disease, disorder, or condition can be selected from retinitis pigmentosa (RP), Leber's congenital amaurosis (LCA), Stargardt disease, Usher's syndrome, choroideremia, a rod-cone or cone-rod dystrophy, a ciliopathy, a mitochondrial disorder, progressive retinal atrophy, a degenerative retinal disease, age related macular degeneration (AMD), wet AMD, dry AMD, geographic atrophy, a familial or acquired maculopathy, a retinal photoreceptor disease, a retinal pigment epithelial-based disease, diabetic retinopathy, cystoid macular edema, uveitis, retinal detachment, traumatic retinal injury, iatrogenic retinal injury, macular holes, macular telangiectasia, a ganglion cell disease, an optic nerve cell disease, glaucoma, optic neuropathy, ischemic retinal disease, retinopathy of prematurity, retinal vascular occlusion, familial macroaneurysm, a retinal vascular disease, an ocular vascular diseases, a vascular disease, and/or ischemic optic neuropathy.

Also provided are methods of screening for agents that affect RPE cell function, proliferation, maturation, differentiation, or survival the method by: a) contacting a population of RPE cells prepared according to any of the methods described herein with at least one agent; and b) determining if the agent has an effect on RPE cell function, proliferation, maturation differentiation, or survival. In various embodiments, the at least one agent is a biological agent (e.g., consisting of a growth factor, a trophic factor, a regulatory factor, a hormone, an antibody or an antigen-binding fragment thereof, a small molecule, and/or a peptide).

Additionally, the disclosure also provides in vitro methods for examining the role of RPE cells in retinal development by: a) preparing an RPE monolayer culture according to any of the methods described herein; and b) monitoring the function, proliferation, maturation, differentiation, survival, or any combination thereof of cells within the RPE monolayer culture during retinal development. For example, in such methods, the monitoring provides information regarding normal retinal development and/or information regarding retinal abnormal development, diseases, disorders, or conditions (e.g., information regarding underlying mechanisms of retinal abnormal development, diseases, disorders, and/or conditions).

Any of the aspects and embodiments described herein can be combined with any other aspect or embodiment as disclosed here in the Summary of the Invention, in the Drawings, and/or in the Detailed Description of the Invention, including the below specific, non-limiting, examples/embodiments of the present invention.

Unless otherwise defined, all technical and scientific terms used herein have the same meaning as commonly understood by one of ordinary skill in the art to which this application belongs. In the specification, the singular forms also include the plural unless the context clearly dictates otherwise.

Although methods and materials similar to or equivalent to those described herein can be used in the practice and testing of the application, suitable methods and materials are described below. All publications, patent applications, patents, and other references mentioned herein are incorporated by reference.

The references cited herein are not admitted to be prior art to the claimed application. In the case of conflict, the present specification, including definitions, will control. In addition, the materials, methods, and examples are illustrative only and not intended to be limiting.

Other features and advantages of the application will become apparent from the following detailed description in conjunction with the examples.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A, FIG. 1B, FIG. 1C, FIG. 1D, FIG. 1E, FIG. 1F, FIG. 1G, FIG. 1H and FIG. 1I show the isolation and derivation of induced-primary retinal pigment epithelial (ipRPE) cultures from human three-dimensional (3D) retinas. FIG. 1A shows the retinal pigment epithelial cells (RPE) that are dissected from 3D retinas. FIG. 1B, FIG. 1C, FIG. 1D, FIG. 1E and FIG. 1F show clusters of RPE cells isolated from the 3D retinas that were collected and dissociated into single cells for culture. Under these conditions, they develop into an RPE monolayer showing a similar behavior to that observed in human primary RPE cultures. FIG. 1G, FIG. 1H and FIG. 1I show images of this RPE monolayer.

FIG. 2A, FIG. 2B, FIG. 2C, FIG. 2D, FIG. 2E, FIG. 2F, FIG. 2G, FIG. 2H, FIG. 2I and FIG. 2J show the characterization of the RPE derived from 3D retinas. FIG. 2A shows the RPE dissected from 3D retinas, dissociated into single cells and seeded on transwells. FIG. 2B shows that the RPE forms a monolayer with the distinctive pigmented cobblestone pattern. FIG. 2C, FIG. 2D, FIG. 2E, FIG. 2F, FIG. 2G, FIG. 2H and FIG. 2I shows that the RPE forms proper ultrastructural differentiation. FIG. 2D and FIG. 2E show that passage two ipRPE cultures show the cobblestone pattern. FIG. 2F and FIG. 2G show the expression of functional proteins in passage two ipRPE cultures. FIG. 2H and FIG. 2I show that passage two ipRPE cultures show polarization with formation of tight junctions and microvilli with appropriate subcellular localization of ZO1 and EZRIN. FIG. 2J shows that the passage two ipRPE cultures show transepithelial resistance comparable to that observed in human fetal primary RPE cultures.

FIG. 3A, FIG. 3B, FIG. 3C and FIG. 3D show the characterization of the RPE derived from 3D retinas. The ipRPE cultures are amenable to sequential passages while maintaining their RPE identity. FIG. 3A, FIG. 3B and FIG. 3C show gene and protein expression in ipRPE cultures obtained from four different passages (P1-P4). FIG. 3C and FIG. 3D show polarized release of VEGF-A in ipRPE cultures measured in the apical and basal extracellular media ipRPE monolayers grown on transwell inserts after different passages.

FIG. 4A, FIG. 4B, FIG. 4C, FIG. 4D, FIG. 4E and FIG. 4F show 3D retinas obtained from hiPSC. FIG. 4A shows that hiPSC form 3D retinas composed of a neural retina (NR) and RPE bundled at the tip. FIG. 4B, FIG. 4C and FIG. 4C show that the NR shows the characteristic layers, including a rod-enriched ONL. FIG. 4D and FIG. 4F show that photoreceptors achieve advanced morphological, molecular and ultrastructural differentiation, including the formation of outer segments (arrowheads) and light response.

FIG. 5A, FIG. 5B, FIG. 5C and FIG. 5D show a stem cell-derived retinal/RPE transplant. FIG. 5A and FIG. 5B show representative light microscopy images of a top view (FIG. 5A) and a bottom view (FIG. 5B), showing physical association between retina and RPE. The transparent appearance of the retina in FIG. 5A reflects its healthy status. FIG. 5C shows a 3D reconstruction of 20 consecutive image planes (5 μm depth spacing) that allowed measurement of the thickness of the retinal/RPE transplant. FIG. 5D is a 3D rendering on a retinal/RPE transplant labeled with Hoechst (RPE) and SYTO green (retina).

FIG. 6A, FIG. 6B, FIG. 6C, FIG. 6D, FIG. 6E, FIG. 6F, FIG. 6G, FIG. 6H, FIG. 6I and FIG. 6J show the generation of rod-enriched vs. cone-enriched 3DNR. FIG. 6A shows that 3DNR of 150 days of differentiation show well organized ONL with advanced differentiated photoreceptors. FIG. 6B shows that by fine-tuning the composition of the culture media (e.g., the retinoic acid (RA) regime) during early differentiation, it is possible to generate rod-enriched 3DNR. FIG. 6C and FIG. 6D show that by fine-tuning the composition of the culture media (e.g., the retinoic acid (RA) regime) during early differentiation, it is possible to generate cone-enriched 3DNR. FIG. 6E and FIG. 6F show that upon further differentiation, retinal bipolar precursors generate all bipolar cell types, including Rod Bipolar cells (RB: Chx10+PKCα+/Islet1+). FIG. 6G, FIG. 6H and FIG. 6I show that upon further differentiation, retinal bipolar precursors generate all bipolar cell types, including Cone OFF bipolar cells (OFF-CB: Chx10+/Scgn+/Islet1− (arrowhead)) and Cone ON bipolar cells (ON-CB: Chx10+/Scgn−/Islet1+ (arrow)). FIG. 6J shows SV2 expression demarcating a developing outer plexiform layer.

### DETAILED DESCRIPTION OF THE INVENTION

### Definitions

In this disclosure, “comprises,” “comprising,” “containing,” “having,” and the like can have the meaning ascribed to them in U.S. Patent law and can mean “includes,” “including,” and the like; the terms “consisting essentially of” or “consists essentially” likewise have the meaning ascribed in U.S. Patent law and these terms are open-ended, allowing for the presence of more than that which is recited so long as basic or novel characteristics of that which is recited are not changed by the presence of more than that which is recited, but excludes prior art embodiments.

Unless specifically stated or obvious from context, as used herein, the terms “a,” “an,” and “the” are understood to be singular or plural.

Unless specifically stated or obvious from context, as used herein, the term “or” is understood to be inclusive.

As used herein, the term “about,” unless indicated otherwise, refers to the recited value, e.g., amount, dose, temperature, time, percentage, etc., ±10%, ±9%, ±8%, ±7%, ±6%, ±5%, ±4%, ±3%, ±2%, or ±1%.

As used herein, the terms “patient” or “subject” are used interchangeably herein to refer to any mammal, including humans, domestic and farm animals, and zoo, sports, and pet animals, such as dogs, horses, cats, and agricultural use animals including cattle, sheep, pigs, and goats. One preferred mammal is a human, including adults, children, and the elderly. A subject may also be a pet animal, including dogs, cats and horses. Preferred agricultural animals would be pigs, cattle and goats.

The terms “treat”, “treating”, “treatment” and the like, as used herein, unless otherwise indicated, refer to reversing, alleviating, inhibiting the process of, or preventing the disease, disorder or condition to which such term applies, or one or more symptoms of such disease, disorder or condition and includes the administration of any of the compositions, pharmaceutical compositions, or dosage forms described herein, to prevent the onset of the symptoms or the complications, or alleviating the symptoms or the complications, or eliminating the disease, condition, or disorder. Preferably, treatment is curative or ameliorating.

As used herein, “preventing” means preventing in whole or in part, or ameliorating or controlling, or reducing or halting the production or occurrence of the thing or event, for example, the disease, disorder or condition, to be prevented.

The phrases “therapeutically effective amount” and “effective amount” and the like, as used herein, indicate an amount necessary to administer to a patient, or to a cell, tissue, or organ of a patient, to achieve a therapeutic effect, such as an ameliorating or alternatively a curative effect. The effective amount is sufficient to elicit the biological or medical response of a cell, tissue, system, animal, or human that is being sought by a researcher, veterinarian, medical doctor, or clinician. Determination of the appropriate effective amount or therapeutically effective amount is within the routine level of skill in the art.

The terms “administering”, “administer”, “administration” and the like, as used herein, refer to any mode of transferring, delivering, introducing, or transporting a therapeutic agent to a subject in need of treatment with such an agent. Such modes include, but are not limited to, intraocular, oral, topical, intravenous, intraperitoneal, intramuscular, intradermal, intranasal, and subcutaneous administration.

The terms “RPE” and “ipRPE” and the like are used interchangeably herein to refer to retinal pigment epithelial cells cultured according to any of the methods described herein and/or used in the three-dimensional tissue products described herein.

The terms “human retinal organoid”, “3DNR”, “three dimensional neuro retina” and the like are used interchangeably herein to refer to the retina.

The terms “retina/RPE transplant” and “3DNR/RPE transplant” and the like are used interchangeably herein to refer to any of the three-dimensional tissue products described herein.

### Retinal Development and Formation of Retinal Organoids

Retinal development occurs within a very dynamic and complex microenvironment involving highly-coordinated cell-cell interactions through direct contact or diffusible signals. (See Adler et al., Dev Biol 305:1-12 (2007); Bassett et al., Trends in Neurosciences 35:5650573 (2012)). Previous work demonstrated that hiPSCs can be induced to differentiate into retinal progenitors that self-organized into a three-dimensional retinal cup using a simple procedure. (See US 2016/033312, which is herein incorporated by reference in its entirety).

Eye development in the embryo's neural plate begins with the formation of the eye field (EF), a centrally-organized domain consisting of a subpopulation of anterior neuroepithelial cells that have become further specified into retinal progenitors. The EF is characterized by the expression of a group of transcription factors including PAX6, RX, LHX2, SIX3, and SIX6, while the surrounding anterior neuroepithelial cells express PAX6 and SOX1. (See Zuber, Curr Top Dev Biol 93:29-60 (2010); Zhang et al., Cell Stem Cell 7:90-100 (2010); Peny et al, Development 125:1967-78 (1998)). In parallel to the native events, hiPSC-derived aggregates, after 8 days of differentiation (D8) in a chemically-defined neural-differentiation medium and attached on Matrigel-coated culture dishes, acquired an anterior-neuroepithelial fate expressing PAX6 and SOX1. Soon after, retinal progenitor cells expressing LHX2 appeared in the central region of the differentiating aggregates. By D12, well-defined EF-like domains expressing the appropriate transcription factors could be observed surrounded by anterior-neuroepithelium-like cells. These anterior-neuroepithelium-like cells typically formed rosettes, which although not found in the native situation, are characteristic of these cells in culture. (See Xia et al., Methods Mol Biol 549:51-58 (2009)).

The EF in vivo gives rise to the left and right optic vesicles, with their respective retinal progenitors eventually forming the future neural retina (NR) and retinal pigment epithelium (RPE). Cell-fate specification into either NR or RPE is regulated critically by two transcription factors, VSX2 and MITF, which initially are co-expressed in the bipotential progenitor cells but subsequently become restricted to the NR and RPE, respectively. (See Adler et al., Dev Biol 305:1-13 (2007); Nguyen et al., Development 127:3581-3591 (2000); Horsford et al., Development 132:177-187 (2005)). Cells within the EF-like domains in our cultures followed the same differentiation sequence. Between D17 and D25 in culture, these NR and RPE domains transitioned to an optic-cup-like structure, with the NR progressively acquiring a horseshoe-dome shape reminiscent of the inner wall of the optic cup, surrounded by the RPE.

In these cultures, retinal progenitors in the EF domains underwent spontaneous differentiation into NR and RPE efficiently and reproducibly, closely mimicking their in vivo topological organization in the correct temporal sequence.

The optic-cup-like shape of the NR domains in the cultures made them easily identifiable and amenable to mechanical detachment one by one, and collection for further culture in suspension. The NR domains, collected in D21-D28, had a high enrichment of NR progenitors and, when cultured in suspension, formed 3-D retinal cups. The retinal cup comprised a thick, transparent NR continuous with the adjacent RPE, which appeared bundled at the tip of the retinal cup and became gradually pigmented. From the time of NR-domain collection to D35 (Week 5, or W5), the NR presented molecular and histological features resembling the actual features of the human embryonic retina at the same age (see O'Rahilly et al., Developmental Stages in Human Embryos (Carnagie Institution of Washington) (1987)), including a polarized, pseudostratified epithelium with proliferating cells undergoing interkinetic nuclear migration and expressing the appropriate transcription factors. During W5-W7, the NR cells spontaneously began to differentiate, following the characteristic center-to-periphery wave of neurogenesis and migrating to their corresponding retinal layers.

### Summary of Prior Methods Used to Generate Stem Cell-Derived RPE Monolayer Cultures

Most prior methods are based on differentiation of stem cells in adherent conditions. (See Table 1). Specifically, stem cells are plated, cultured in the presence of growth factors (or without specific growth factors), morphogenes, or modulators (i.e., agonists and/or antagonists) to induce RPE differentiation. Such methods generate a mixed population of cells, including islands or patches of RPE cells. Following culture, RPE (pigmented) patches are manually picked, expanded and enriched until achieving RPE monolayers.

Another group of prior methods is based on an initial step consisting of embryoid bodies (floating aggregates of mixed cell populations). (See Table 2). In such methods, stem cells are first differentiated into embryoid bodies and cultured for some time. Then, embryoid bodies are plated on adherent conditions, and differentiating cells grow out of the embryoid bodies and differentiate into patches of RPE, which are manually picked expanded and enriched until forming an RPE monolayer.

Finally, a third group of methods is based on an initial step consisting on optic-vesicle or retinal organoid differentiation. (See Table 3). For example, in Meyer et al., Stem Cells 29(8):1206-18 (2011), stem cells are first differentiated into 3D optic vesicles structures and then treated with Activin A for RPE cell differentiation. 3D pigmented vesicles are then plated, and RPE cells grow out of the optic vesicles and form an RPE monolayer. In Wu et al., Oncotarget 7(16):22819-33 (2016), stem cells are first differentiated into neurospheres containing optic vesicles. Following long time culture, optic vesicles develop RPE pigmented clumps or foci that are excised and plated. RPE cells grow out of the foci and form an RPE monolayer

TABLE 1
Prior methods for generating human stem cell-derived RPE culture using adherent conditions throughout the differentiation process

| | | | | | Cell | First RPE/ | | | | | |
|---|---|---|---|---|---|---|---|---|---|---|---|
| | | | | | density | Characterization | TER | PH | | | VEGF/ |
| Author | Cell type | Passage | Chamber | Coating | (cells/cm²) | (Days) | (Ω cm²) | assay | TEM | ICC | PEDF |
| Klimanskaya et al. 2004 | hESC | 1-9 | R | GN | N/A | 21-56/30 | | X | X | | |
| Buchholz et al 2009 | hiPSC/hESC | 0-2 | R | GN | 6.3 × 10⁴ | 20-35/30 | | X | | | |
| Meyer et al. 2009 | hiPSC/hESC | Not | R | LN | N/A | 30-35/30 | | | | | |
| Zahabi et al 2012 | hiPSC/hESC | 1 | R | MG | N/A | 25/40-60 | | | | | |
| Buchholz et al 2013 | hiPSC/hESC | 1 | TW/R | MG | N/A | 14/30 | | X | | | |
| Maruotti et al. 2013 | hiPSC/hESC | 1-3 | TW/R | VN- | 1 × 10⁵ | 33-38/50 | | X | | | X |
| | | | | PAS/MG | | | | | | | |
| Singh et al 2013 | hiPSC | 0-3 | TW/R | LN | 1.4 × 10⁵ | 30/60 | X | X | | X | X |
| Ferrer et al 2014 | hiPSC/hESC | 1-3 | TW/R | MG | 3.8 × 10⁵ | 25-35/42-56 | X | | X | X | |
| Reichman et al. 2014 | hiPSC | 0-2 | R | MEF/GN | N/A | 7/14 | X | | | | |
| Croze et al 2014 | hiPSC/hESC | 1-14 | TW/R | MG | 1 × 10⁵ | 28-42/45 | | X | | | X |
| Leach et al 2015 | hESC | 0-3 | TW/R | MG | N/A | 14-32 | | X | | | X |
| Maruotti et al. 2015 * | hiPSC/hESC | 1 | TW/R | VN- | 2.5-3 × 10⁵ | 28/35 | | X | | | X |
| | | | | PAS/MG | | | | | | | |
| Lidgerwood et al 2016 * | hiPSC/hESC | 0-2 | R | MG | 7.5 × 10⁴ | 20/60 | | X | | | |

TABLE 2
Prior methods for generating human stem cell-derived RPE culture using embryoid bodies at a first step, and adherent conditions thereafter

| | | | | | Cell | | | | | | |
|---|---|---|---|---|---|---|---|---|---|---|---|
| | | | | | density | First RPE/ | TER | PH | | | VEGF/ |
| Author | Cell type | Passage | Chamber | Coating | (cells/cm²) | Characterization | (Ω cm²) | assay | TEM | ICC | PEDF |
| Klimanskaya et al. 2004 | hESC | 1-9 | R | GN | N/A | 28-56/30 | | X | X | | |
| Osakada et al 2008 | hESC | Not | R | PDL/LN/ | N/A | 50/50-120 | | | X | | |
| | | | | FN | | | | | | | |
| Vugler et al. 2008 | hESC | 1-2 | R | MG | 10 | 7-21/30 | | | X | | |
| | | | | | pigmented | | | | | | |
| | | | | | foci | | | | | | |
| Idelson et al. 2009 | hESC | 1-2 | R | PDL/LN | 30-50 | 28/21-35 | | X | X | | |
| | | | | | clusters | | | | | | |
| Vaajasaari et al. 2011 | hiPSC/hESC | Not | TW/R | C-IV | N/A | 10-21/28 | X | X | | | X ^{†} |
| Zhu et al. 2011 | hESC | 1-6 | TW/R | GN/GX/ | 2 × 10⁵ | 56/28 | X | X | | | X ^{†} |
| | | | | FN | | | | | | | |
| Plaza-Reyes et al. 2016 | hESC | N/A | TW/R | LN-521 | 0.6-1.2 × 10⁴ | 21/35 | X | X | | | X |

TABLE 3
Prior methods for generating human stem cell-derived RPE cultures using optic vesicle-like/re-plating conditions

| | | | | | Cell | | | | | | |
|---|---|---|---|---|---|---|---|---|---|---|---|
| | | | | | density | First RPE/ | TER | PH | | | VEGF/ |
| Author | Cell type | Passage | Chamber | Coating | (cells/cm²) | Characterization | (Ω cm²) | assay | TEM | ICC | PEDF |
| Meyer et al. 2011 | hiPSC/hESC | 0-2 | R | LN | N/A | 40/— | | | | X | |
| Zhong et al. 2014 | hiPSC | Not | R | MG | N/A | 16/16 | | | | | |
| Wu et al. 2016 | hESC | 0-2 | TW/R | MG | 1 × 10⁵ | 19/35-45 | X | X | X | | X ^{†} |

hESC: human embryonic stem cells; hiPSC: human induced pluripotent stem cells; R: regular plate; TW: transwell insert; GN: gelatin; MEF: mouse embryonic fibroblasts; MG: matrigel; VN-PAS: vitronectin peptide-acrylate surface; PLD: poly-D-lysine; FN: fibronectin; LN: laminin; C-IV: Collagenase-IV; GX: geltrex; RPE: retinal pigment epithelium; TER: transepithelial resistance; PH: phagocytosis; TEM: Transmission electron microscopy; ICC: intracellular calcium concentration: ; VEGF: Vascular endothelial growth factor; PEDF: Pigment epithelium-derived factor; N/A: not applicable, not available, or no answer; X: performed;
* One step approach: no selection or manual picking
^{†} Only PEDF;

Isolation and Characterization of Induced-Primary RPE (ipRPE) from Human Retinal Organoids

In contrast to these prior methods, in the culture methods described herein, RPE cells follow a spontaneous process of differentiation without the need of exogenous factors to promote RPE cell fate, differentiation and/or maturation. A pure RPE monolayer is obtained from the first step of this method without the need for manual picking and/or purification/enrichment steps, achieving functional maturation by 30 days in culture. In these methods, stem cells are first differentiated into retinal organoids. As retinal organoids differentiate, they also generate RPE tissue forming a clump or RPE tissue attached to the retinal organoid. Importantly, no exogenous growth factors, morphogenes, or modulators (i.e., agonists and/or antagonists) are used for differentiating retinal organoids and RPE. Rather, these cultures undergo spontaneous differentiation. RPE clumps are excised from the retinal organoids and dissociated into a suspension of single cells. Single cells RPE are seeded into petri dishes and cultured until they form a monolayer of RPE. Again, no exogenous growth factors morphogenes, or modulators (i.e., agonists and/or antagonists) are added here either.

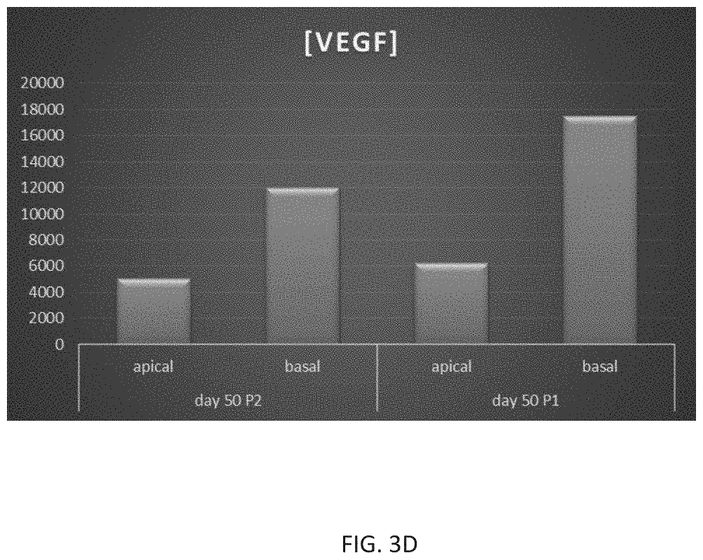

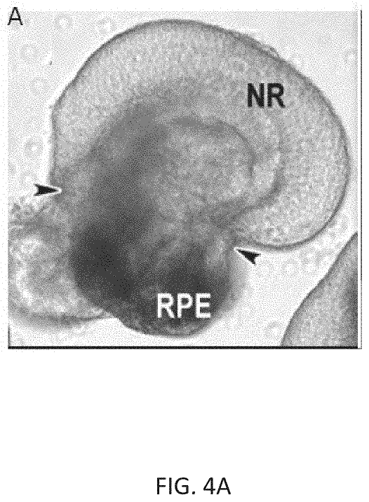

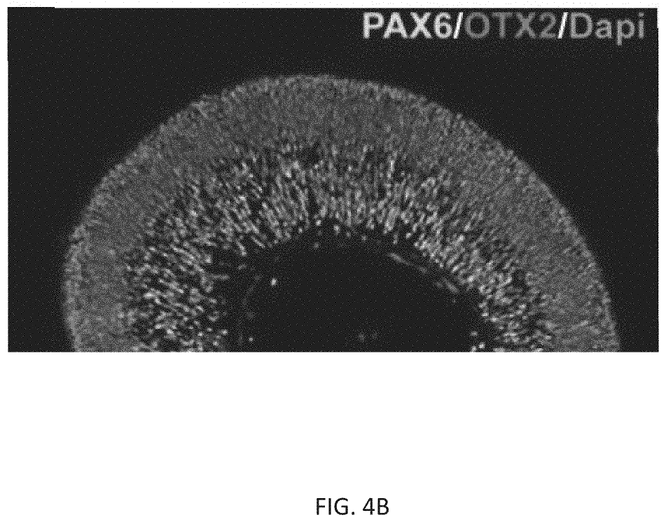

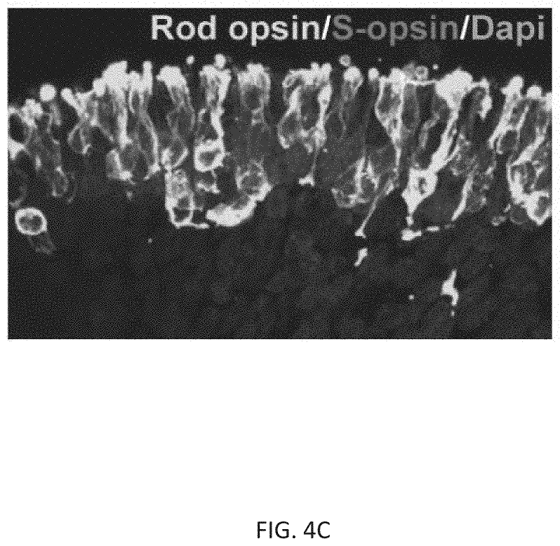

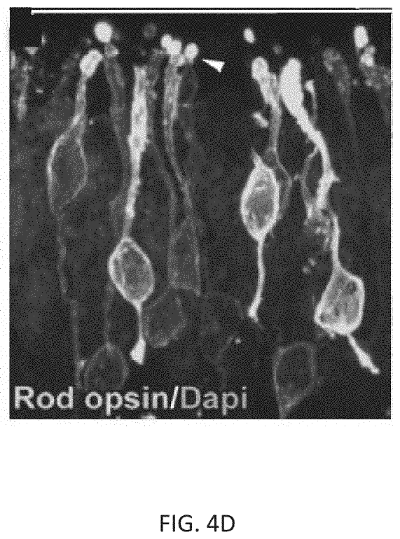

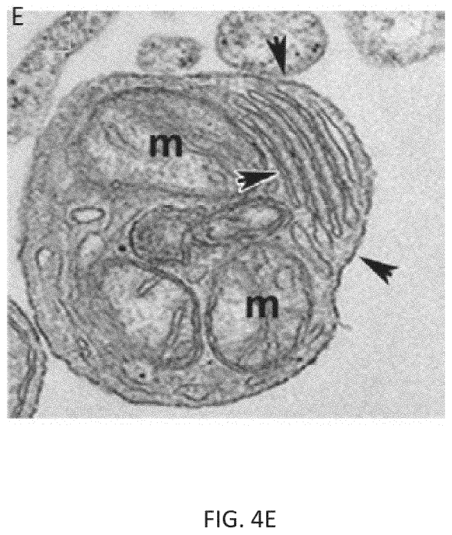

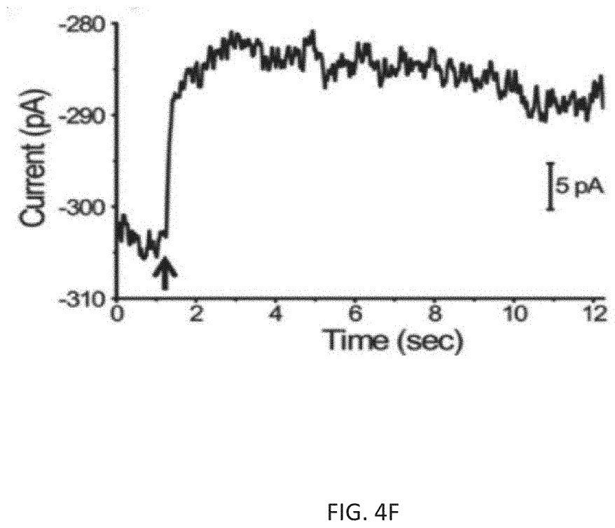

Provided herein is a simple and efficient strategy for isolating and culturing RPE cells from human retinal organoids (hRetOs). Briefly, hRetOs were generated as previously described. (See Zhong et al., Nature Communications 5:4047 (2014) and US Published Patent Application No. 2016/033312, each of which are herein incorporated by reference in its entirety). These hiPSC-derived 3D retinal organoids contain functional photoreceptors, are properly laminated (with a highly organized outer nuclear layer containing advanced differentiated rods and cones (red, green, and blue) displaying inner and outer segments and a light response (see FIG. 4), and show spatial and temporal features that replicate the development of the human retina in vivo. Human iPS cells expressing the pluripotency markers OCT4. Nanog, SEEA1 and Nestin were maintained on Matrigel coated plates.

On day 0 (D0) of differentiation, iPS cell colonies were detached, mechanically dissociated into small clumps and cultured in suspension to induce aggregate formation. Aggregates were gradually transitioned into neural-induction medium to induce anterior neural differentiation. On D7, neural aggregates were seeded onto Matrigel coated dishes.

On D14 after differentiation was initiated, domains expressing neural retinal progenitor markers began to appear, with non-pigmented cells with a typical RPE cobblestone and expressing MITF surrounding these neuroretina (NR) domains. These NR domains gradually acquire a horseshoe-shape with MITF expressing RPE cells surrounding them.

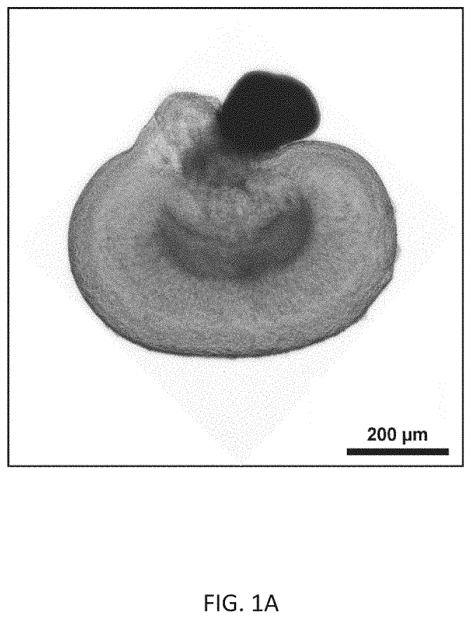

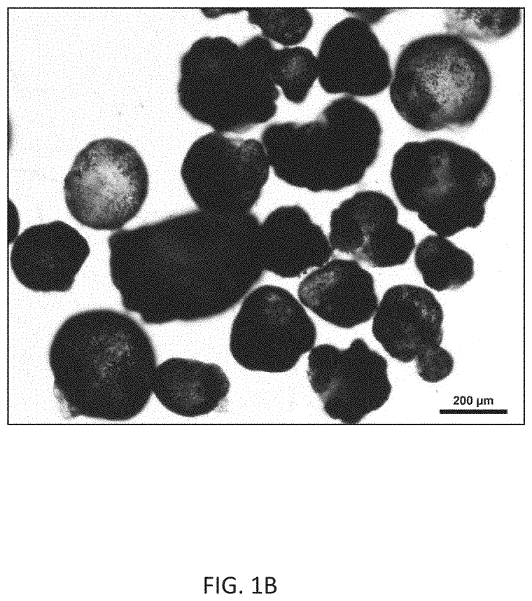

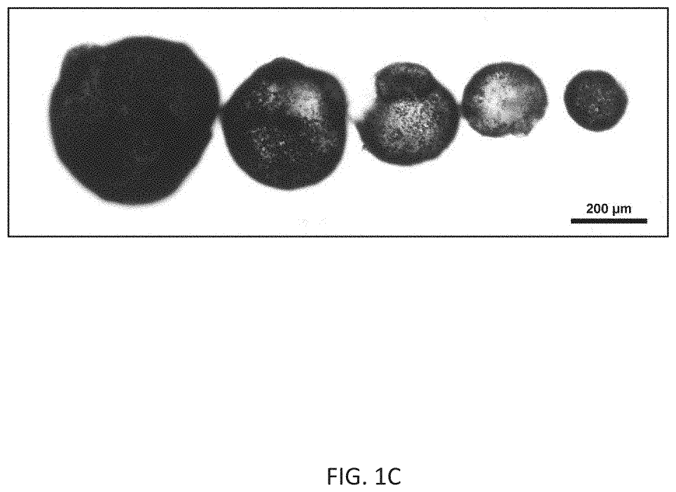

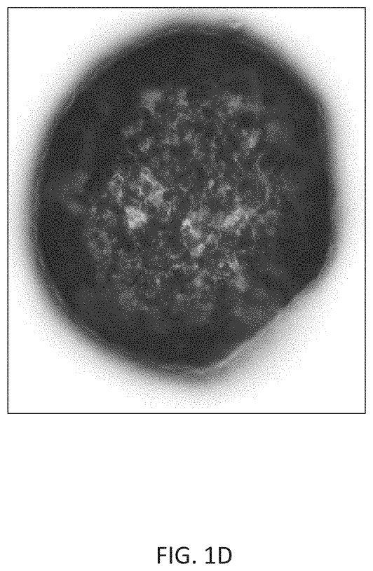

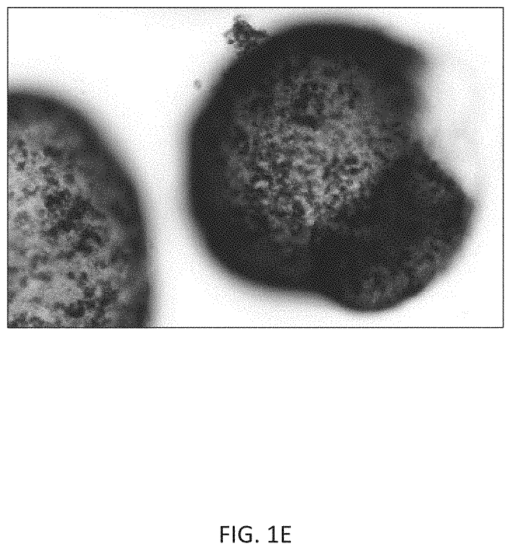

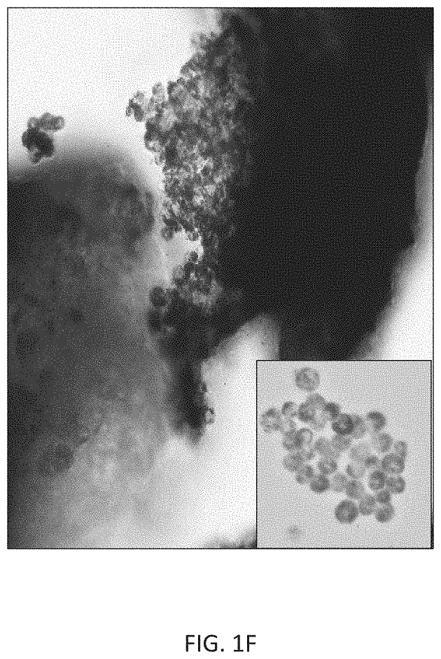

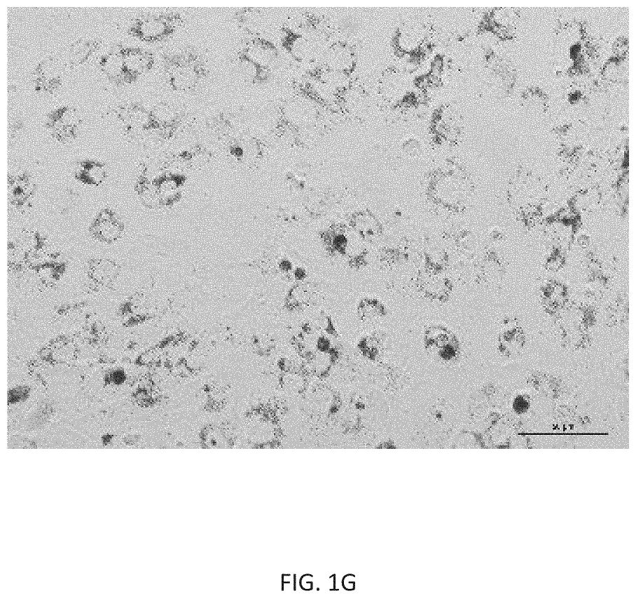

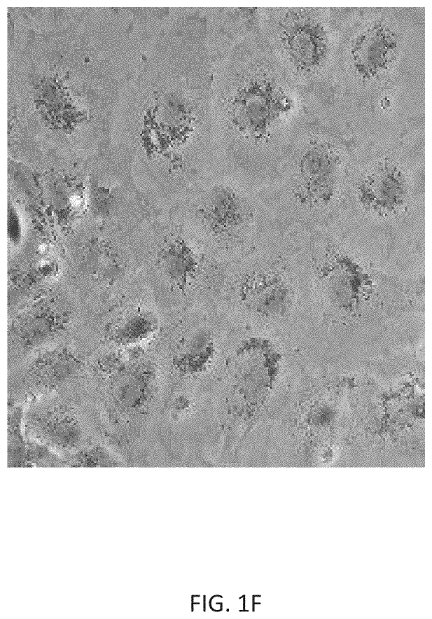

On D21, individual mechanical detachment and collection of the horseshoe-shaped NR and RPE domains is performed, and upon further culture in suspension they gradually form 3D retinal tissue. On D30, the 3D retinal tissue appears fully folded into a 3D retinal organoid and continues resembling the actual features of the human embryonic retina at the same age. The RPE tissue attached to the 3D retinal organoid is observed as early as day 25 to 30 of differentiation. (See FIG. 1).

Between D25 to D50, the RPE attached to the 3D retinal organoids becomes polarized, pigmented, and expressed some of the RPE key markers such as: RPE65 (isomerohydrolase critical for the regeneration of the visual pigment); BEST1 (a calcium-activated anion channel); OTX2 (a transcription factor essential for the development and the maintenance of the RPE); and/or EZRIN (a protein localized in the apical processes).

The pigmented RPE was mechanically isolated from the 3D retinal organoids (e.g., using a tungsten needle), dissociated into single cells RPE and plated onto transwell filters (semiporous polyester membrane) to obtain polarized RPE monolayers. (See FIG. 1). Using this technique, after 4 weeks of total differentiation, pure pigmented RPE tissue was reproducibly isolated from hiPSC-derived 3D retinal organoids. To increase the yield of isolated RPE tissue, pure pigmented RPE tissue can be isolated at D50 of differentiation.

As used herein, the isolated RPE obtained from the 3D retinal organoids as passage 0 (P0). Isolated RPE is used to generate RPE monolayer cultures, which will be referred as induced-primary RPE monolayers (ipRPE).

Characterization and Development of Human Induced-Primary RPE (ipRPE) Monolayers

The RPE (P0) from hiPSC-derived 3D retinal organoids is isolated and cultured onto transwells (P1) to establish the induced-primary RPE (ipRPE) monolayers. On D1 after plating the ipRPE, pigmentation was initially lost in most of the cells. However, as the cells continued to divide, pigment density increases, indicating the de novo synthesis of pigment. (See FIG. 1). The newly dividing cells retained their epithelioid morphology.

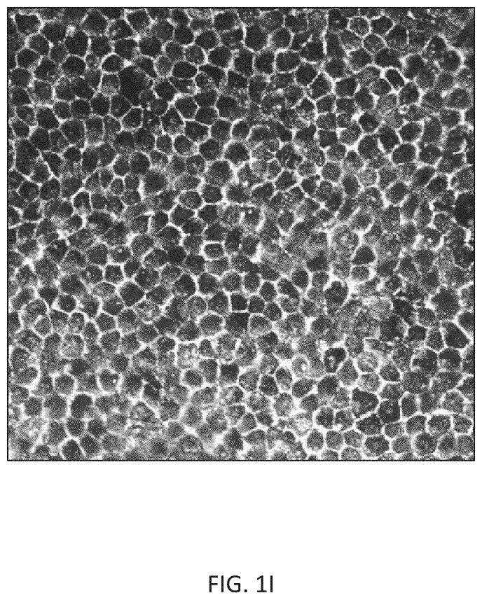

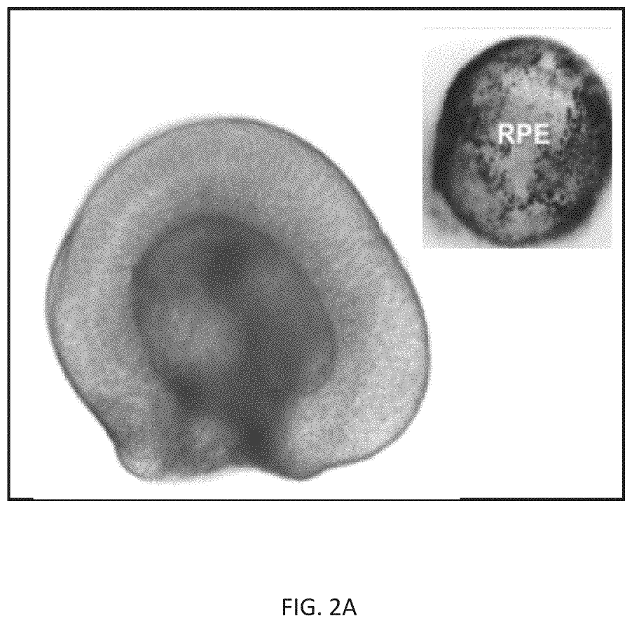

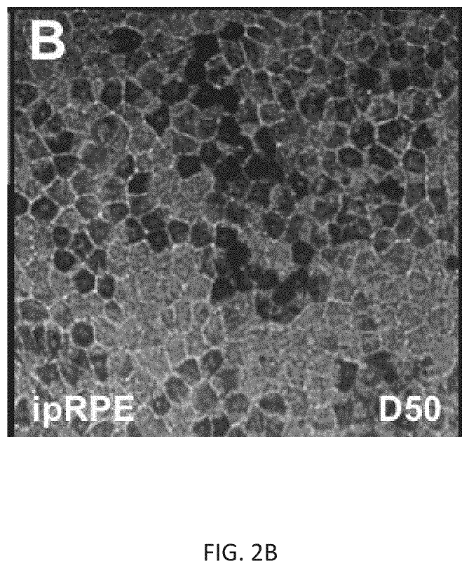

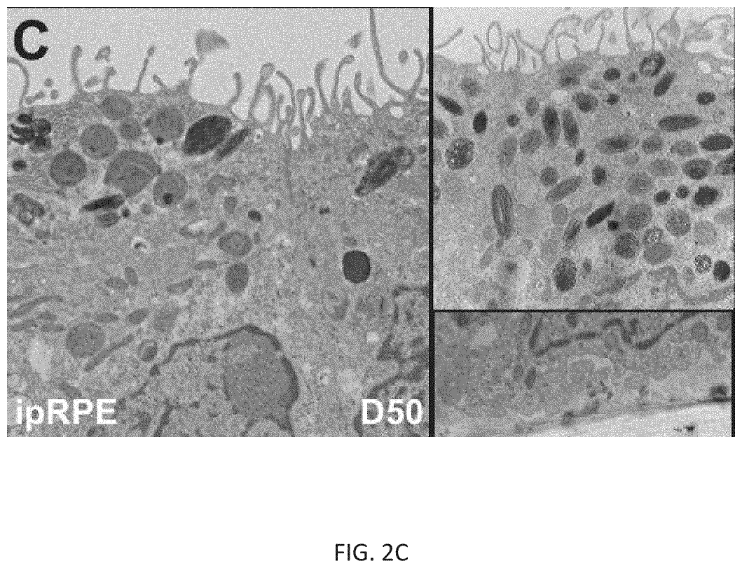

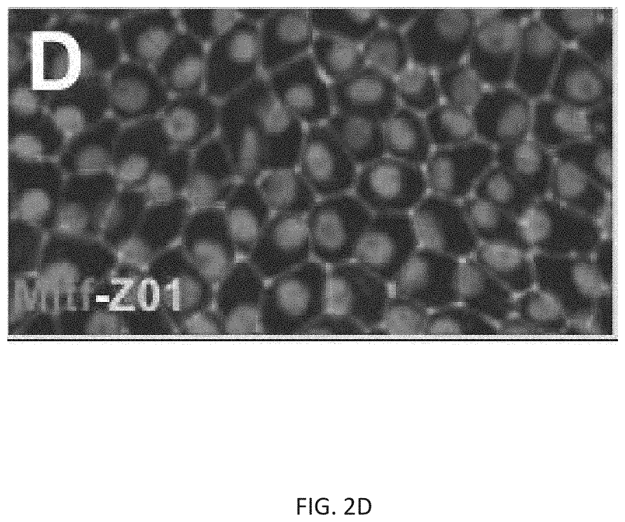

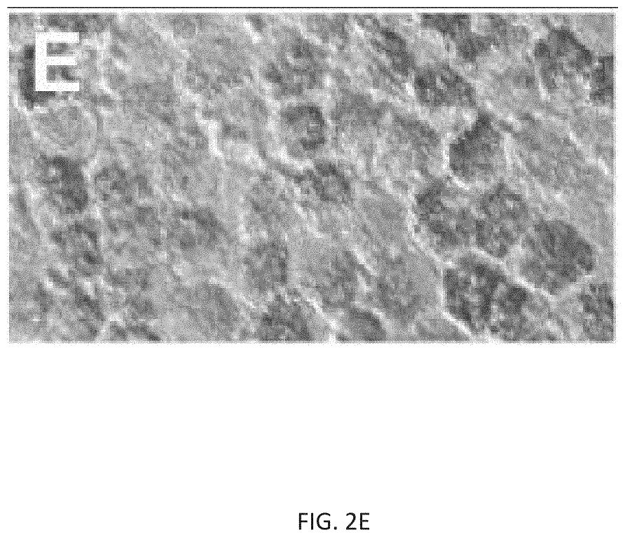

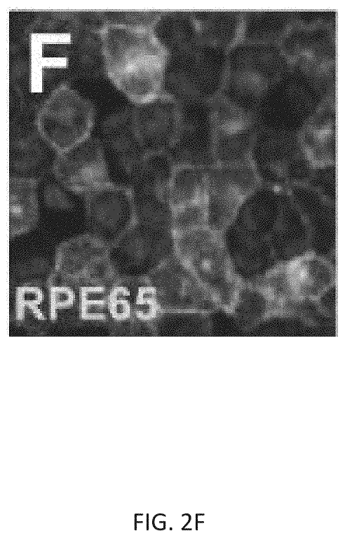

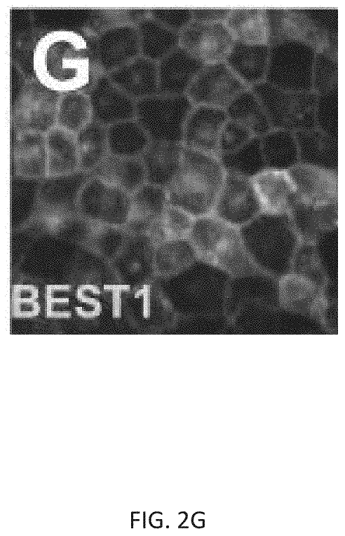

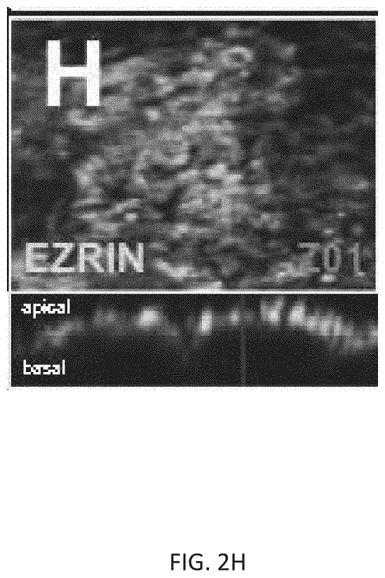

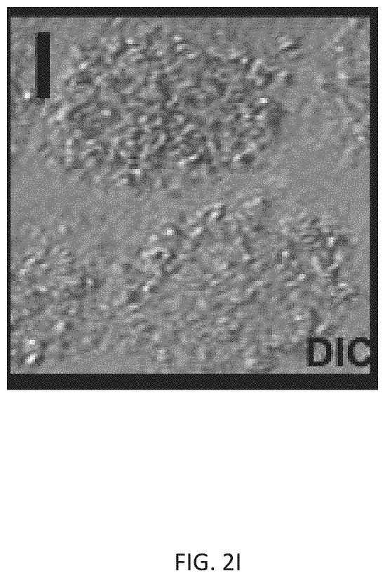

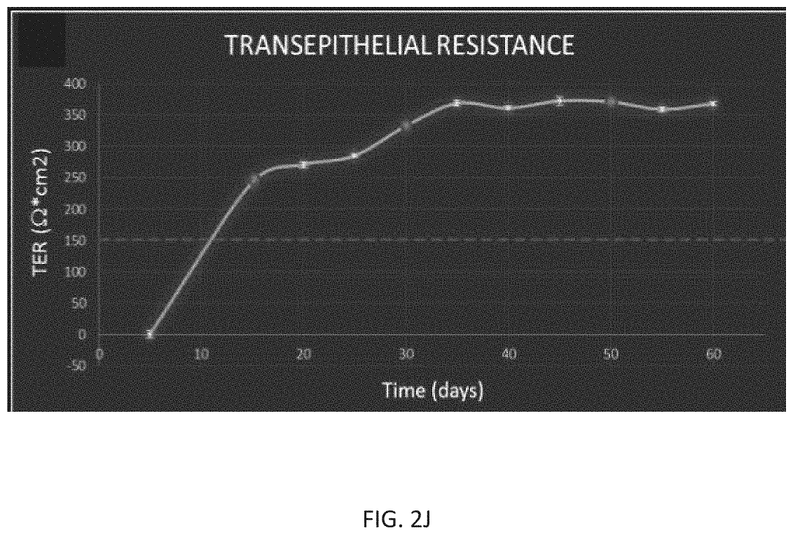

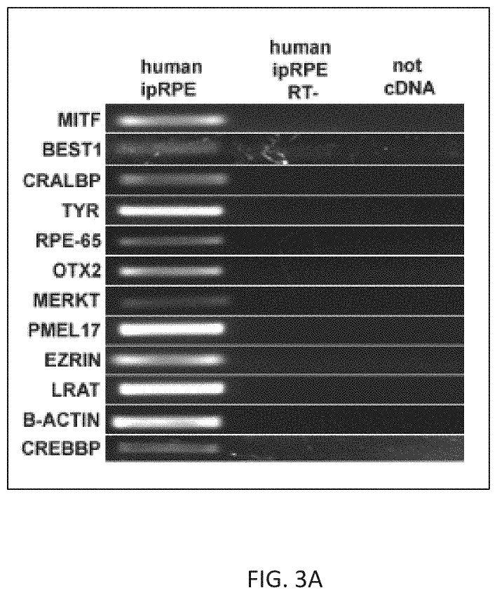

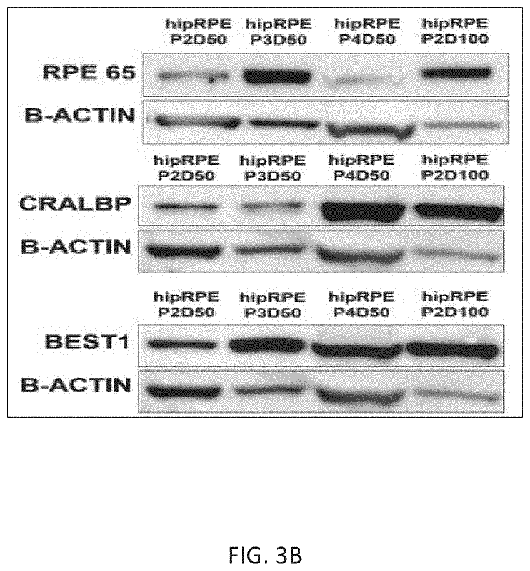

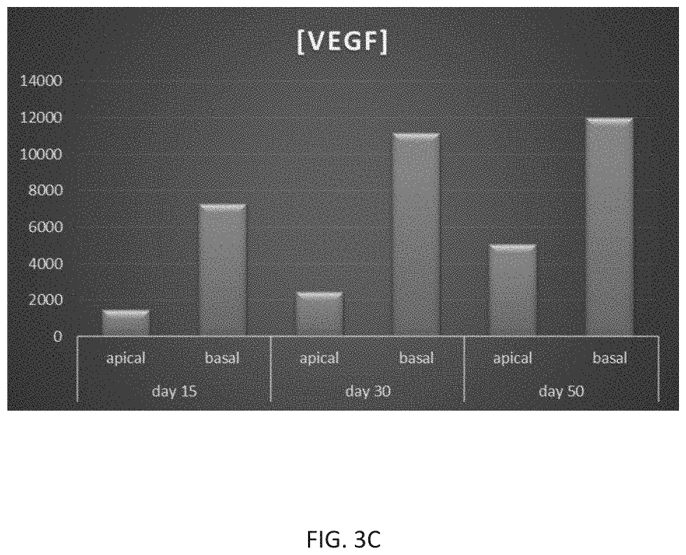

As the cells in culture matured, the characteristic polygonal shape and pigment density became more uniform. (See FIG. 2). Once P1 ipRPE monolayer is established, subsequent passages and expansion of the RPE cells were performed every 10 days. ipRPE monolayers have been characterized from P1 to P4, and it was found that ipRPE monolayers retained the RPE phenotype and expressed the key RPE markers by RT-PCR and western blot in all of the passages. (See FIG. 3). ipRPE cells have been shown to be amenable to sequential passaging retaining their RPE differentiation and maturation capacity until at least passage 4.

In order to use the best passage, P1 and P2 were compared. (See FIG. 3). ipRPE-P1 reached higher transepithelial resistance (TER) levels compared to P2. Both cell passages were capable of polarized release of VEGF. (See FIG. 3). In order to validate ipRPE-P2, the cells were characterized on D50. ipRPE monolayers expressed MITF and RPE65. (See FIG. 2). ZO1 expression on the apical side and BEST1 on the basal side of the cells confirmed a well-polarized RPE monolayer. (See FIG. 2).

The pigmented cells had the structural characteristics of RPE including abundant apical microvilli, adherents junctions, and tight junctions (as evidenced by the measurements of transepithelial resistance, which increases gradually as the ipRPE cells mature until reaching a plateau) observed under transmission electron microscopy. (See FIG. 2). Taken together, these data demonstrate that the ipRPE pigmented monolayer on passage 2 is polarized, functional and expressed the key hallmarks of bona fide RPE cells.

### Uses of RPE Cell Monolayers

RPE cells generated according to the methods of the disclosure can be used in a variety of ways. For example, the cells can be used as a transplant for stem cell-based regenerative therapies for retinal diseases. (See Bharti et al., Invest. Ophthalmolol. Vis Sci 55:1191-1201 (2014); Trounson et al., Cell Stem Cell 17:11-22 (2015)). As no treatment is currently available for retinal diseases such as the dry form of age-related macular degeneration, there is a large potential market for treatments that utilize RPE cells prepared according to any of the methods described herein.

Likewise, these cells can also be used as an in vitro disease model for uncovering disease mechanisms and developing therapies. Alternatively (or additionally), such RPE cells can also be used for drug screening in order to identify agents that influence RPE cell function, proliferation, maturation, differentiation, and/or survival.

### Stem Cell-Derived Retina/RPE Complex

Provided herein is a stem cell-based product consisting of a three-dimensional tissue product biological unit containing integrated 3D retina tissue and RPE tissue. This three-dimensional tissue product is derived from stem cells (e.g., human induced pluripotent stem cells (hiPSCs)) and is composed of functionally matured RPE and differentiated neural retina. Due to the versatility of the co-culture method, stem cell-derived retinas and RPE can be combined at different times of cell maturation.

This three-dimensional tissue product described herein can be distinguished from other products. For example, Eiraku et al., Nature 472(7341):51-6 (2011) and Nakano et al., Cell Stem Cell 10:771-85 (2012) describe the early formation of an optic cup, where the still-undifferentiated neural retina invaginates into an optic cup and gets apposed to the still undifferentiated RPE tissue. This spatial organization is only temporary and the two tissues do not achieve differentiation as a combined complex. Likewise, Zhu et al., PLoS One. 2013; 8(1):e54552 (2013) describes one experiment involving co-culture of hESC-derived RPE cells and mouse retinal explants (explants of retinal tissue obtained directly from the mouse eye). Finally, Yanai et al., Tissue Eng Part A. (11-12):1763-71 (2015) uses a co-culture system involving hESC-derived RPE monolayer and retinal explants from human and rodents (retinal tissue obtained directly from human and rodent eyes).

In contrast, this technology utilizes hiPSC-derived 3D retinas with functional photoreceptors and functionally matured RPE generated from hiPSC-derived 3D retinas in order to generate the stem cell-derived three-dimensional tissue products described herein. hiPSC-derived 3D retinal tissue containing functional photoreceptors are generated in accordance with the methods described in Zhong et al., Nature Communications 5:4047 (2014) and US Published Patent Application No. 2016/033312. These 3D retinas follow the same program and timing of differentiation as the native human retina, beginning with an undifferentiated neural retina epithelium and culminating with a fully laminated retinal tissue. As shown in FIG. 4, these hiPSC-derived 3D retinas achieve proper lamination, with a highly organized outer nuclear layer (ONL) containing advanced-differentiated rods and cones (red, green and blue) displaying inner and outer segments and a light response.

Although RPE cells also differentiate in this system, they do not form a monolayer covering the outer nuclear layer. (See FIG. 4A). Importantly, the RPE cells can be easily dissected away, allowing independent culture of 3D neural retinas (3DNR) and RPE cells. (See FIG. 2). Thus, a new methodology to derive RPE monolayer cultures from our 3D retinas has been established. As described herein, RPE tissue is dissected from the 3D retinas, dissociated into single cells and seeded on transwells where they form characteristic RPE monolayers showing a similar behavior to that observed in human primary RPE cultures. By day 50 of differentiation, the RPE monolayer shows the distinctive pigmented cobblestone pattern, with normal ultrastructural differentiation including formation of specialized functional structures such as microvilli, tight junctions, and basal infoldings (see FIG. 4B-4C), and appropriate subcellular expression and localization of the characteristic genes indicative of a mature state that are also observed in native human RPE cells and primary culture.

hiPSC-derived 3D retina and RPE tissue can be combined to form a functionally integrated complex composed of a layer of neural retina and an underlying layer of RPE cells. Both the retina and RPE layers recreate the cellular and topological organization observed in the normal human retina. This three-dimensional tissue product is the first stem cell-derived system that recreates the physical and functional interactions between the neural retina and RPE that occur in the native retina. Importantly, this product solves the current problem of lack of a stem cell-derived system capable of recreating the physical and functional interactions between the neural retina and RPE.

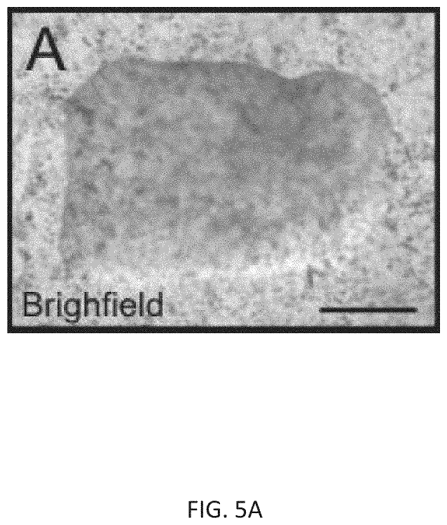

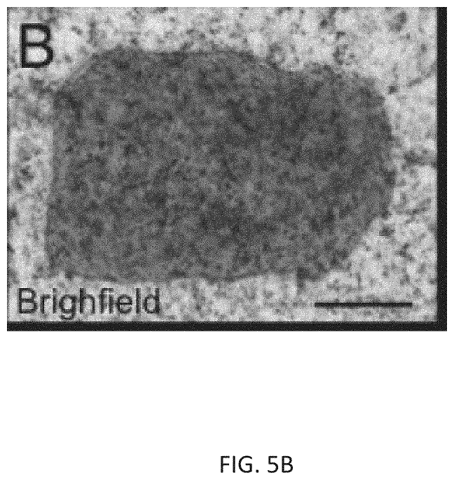

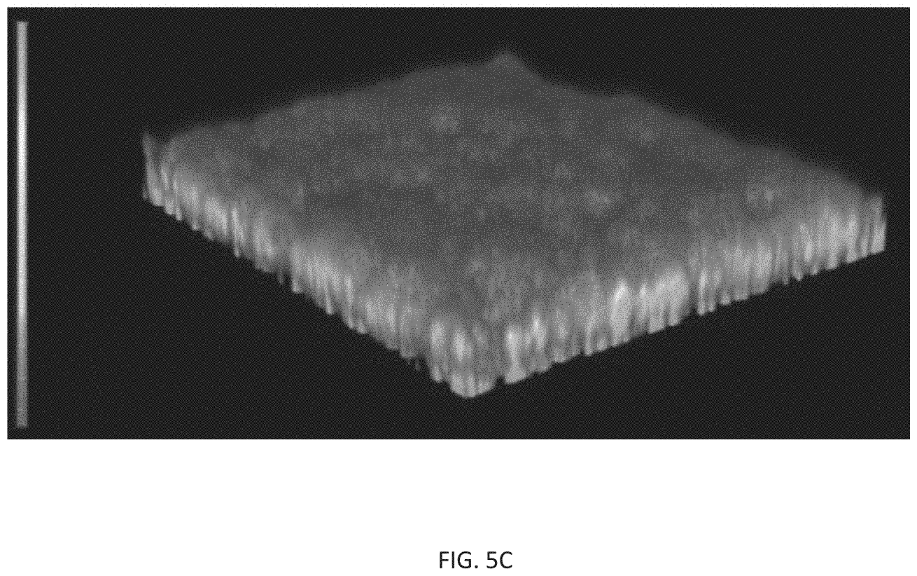

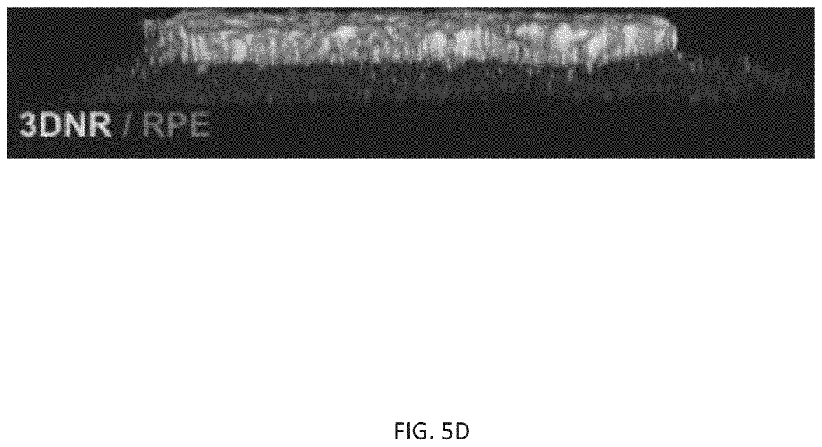

hiPSC-derived 3D retinal tissue is generated as described in Zhong et al., Nature Communications 5:4047 (2014) and US Published Patent Application No. 2016/033312. Neural retinal patches (3DNR) are prepared from the 3D retinas. Specifically, hiPSC-derived 3D retinas are opened (e.g., using a tungsten needle or any other method known in the art) in order to expose the inside of the 3D retinal cups, flattened as a retinal flat mount, and retinal explants are obtained. These retinal explants (or patches) (e.g., approximately 1.5 mm×1.5 mm) are then seeded on top of ipRPE (passage 2) and co-cultured for different periods of time (e.g., 24, 25, 26, 27, 28, 29, 30, 31, 32, 33, 34, 35, 36, 37, 38, 39, 40, 41, 42, 43, 44, 45, 46, 47, 48, or more hours). Under these conditions the 3DNR attach to the RPE monolayer forming a 3DNR/RPE complex. (See FIG. 5). Incorporation of an additional biocompatible component (e.g., a natural or synthetic compound in a liquid or gel form that provides an appropriate biomechanical environment for cell survival and function (e.g., a hydrogel) and/or a biocompatible scaffold (e.g., a natural or synthetic scaffold, a scaffold made from biodegradable materials, a scaffold made from non-biodegradable materials, and/or any combinations thereof) into the system further provides an improved biomechanical environment allowing for longer culture periods and manipulation during transplantation. The inclusion of this additional biocompatible component promotes survival and function of the transplanted cells.

Thus, the three-dimensional tissue products derived from human induced pluripotent stem cells (hiPSCs) described herein contain functionally matured RPE cells and a portion of 3DNR. In embodiments, such three-dimensional tissue products may also contain an additional biocompatible component (i.e., a natural or synthetic compound in a liquid or gel form that provides an appropriate biomechanical environment to promote cell survival and function of the transplanted cells and/or allows for manipulation of the product), and a biocompatible scaffold (i.e., natural or synthetic scaffolds, scaffolds made from biodegradable materials, scaffolds made from non-biodegradable materials, or any combinations thereof), wherein the 3DNR, the RPE cells, and the additional biocompatible component are physically and functionally integrated to form a complex containing a layer of neural retina and an underlying layer of RPE cells. The RPE cells can be grown on top of the biocompatible scaffold prior to integration with the 3DNR, such that the 3DNR is positioned on top of the RPE cells. For example, the 3DNR and RPE may be embedded in the additional biocompatible component.

Those skilled in the art will recognize that the 3DNR can be i) undifferentiated pseudostratified neural retina epithelium; ii) laminated neural retina tissue including all retinal layers and their corresponding retinal precursor cell types; and/or iii) advanced differentiated retinal tissue including an outer nuclear layer (ONL) and a bipolar cell layer (BCL), wherein the ONL could be rod-enriched, cone-enriched, or any combination thereof and/or that the RPE can be i) obtained from the initial plating or any passage thereafter; ii) at early stages of differentiation, and/or iii) at more advanced stages of differentiation, times in culture, or combinations thereof. Any combination(s) of these 3DNR and RPE can be used in any of the three-dimensional tissue products described herein.

RPE cells can be prepared using any of the methods described herein. By way of non-limiting example, RPE cells can be prepared by a) culturing human retinal organoids in a first culture medium that is not supplemented with exogenous growth factors, morphogenes, or modulators of their signaling pathways, to generate RPE cells and neural retina (NR); b) isolating RPE tissue from the cultured retinal organoids; c) dissociating the isolated RPE tissue into a suspension of single RPE cells; d) plating single RPE cells in an adherent culture; and e) culturing the plated cells in a second culture medium that is not supplemented with exogenous growth factors, morphogenes, or modulators of their signaling pathways, to produce a monolayer of RPE.

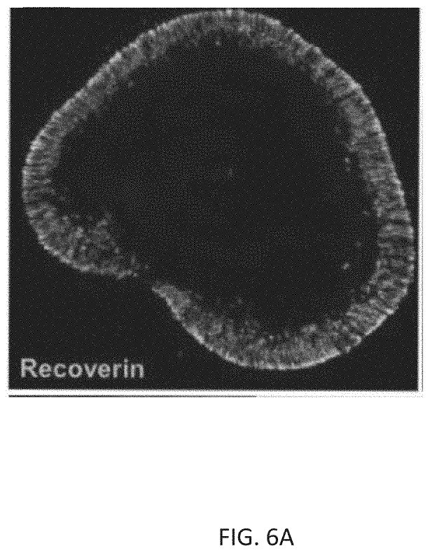

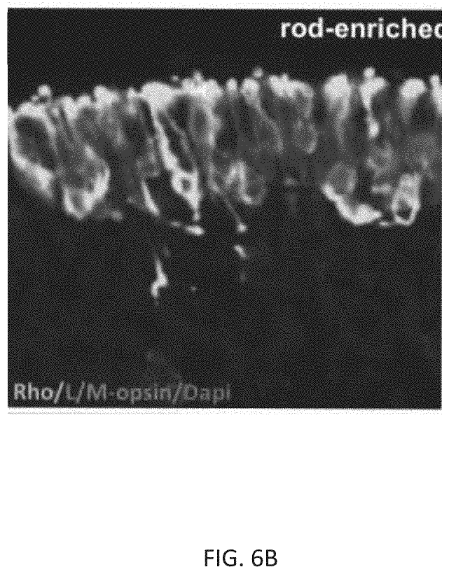

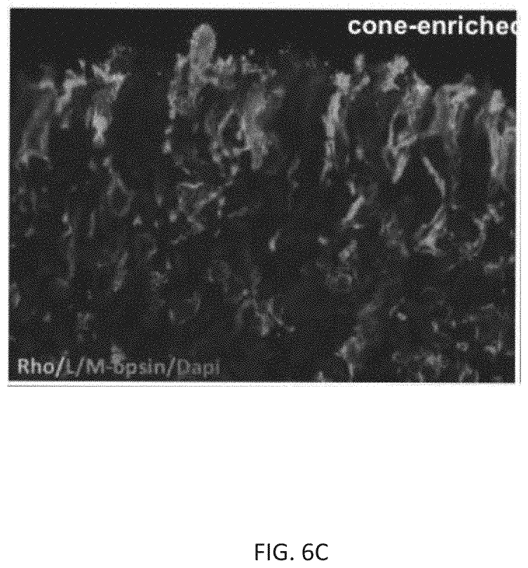

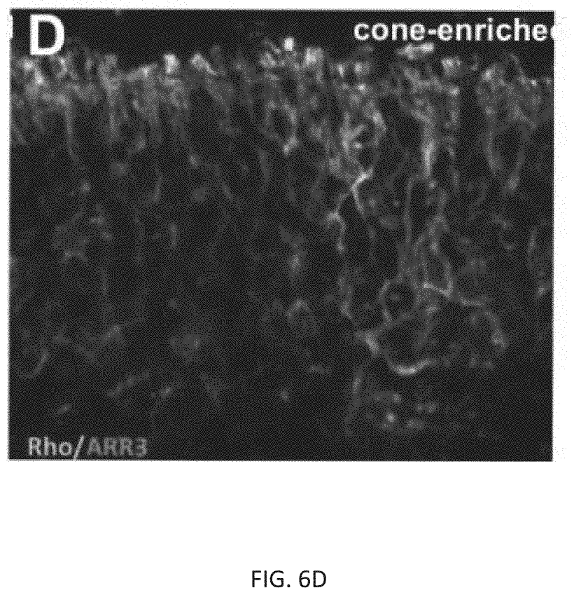

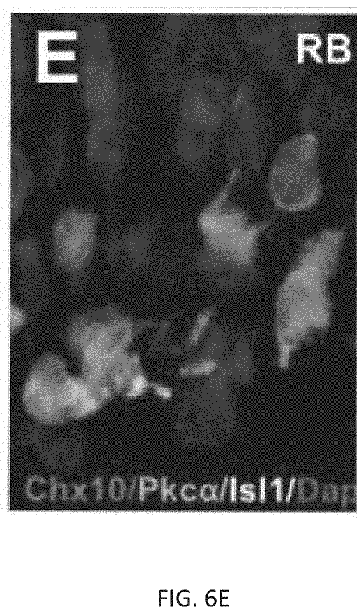

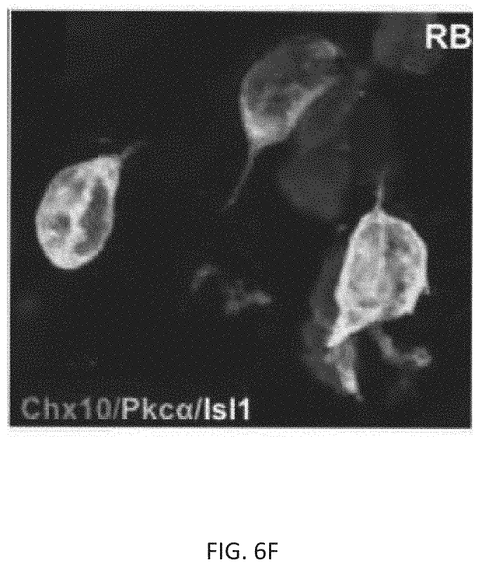

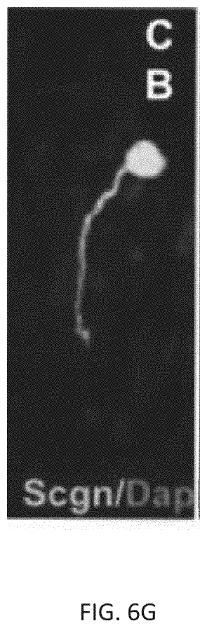

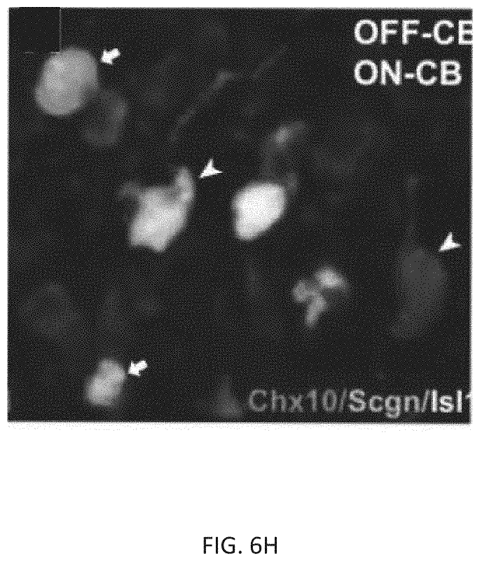

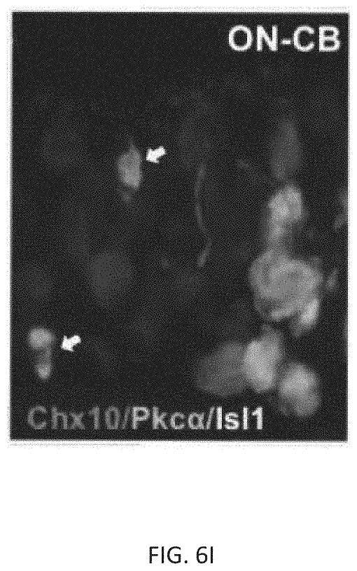

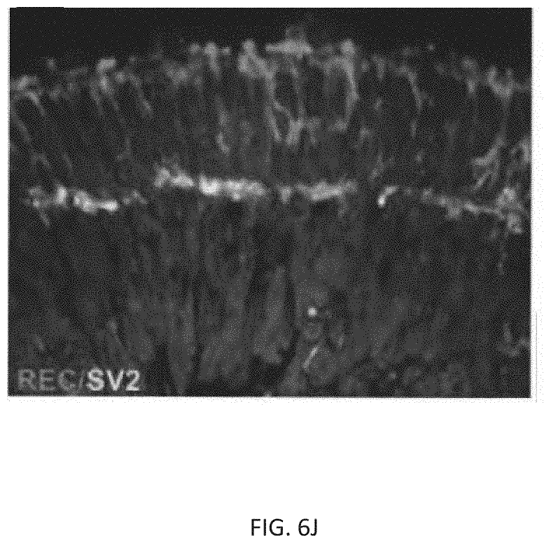

Using the methods descried herein, it is possible to generate both rod-enriched and cone-enriched hiPSC-derived retinal tissue. (See FIGS. 6A-6J). For example, using the retinoic acid (RA) regime described in Zhong et al., Nature Communications 5:4047 (2014), it is possible to reproducibly generate rod-enriched 3DNR. (See also and US Published Patent Application No. 2016/033312). Likewise, modifying the RA regime allows the generation of cone-enriched hiPSC-derived retinal tissue. Additionally, bipolar cells in the 3DNR have the capacity to differentiate into all major bipolar cell subtypes, including rod, cone ON and cone OFF bipolar cells. Moreover, rods and cones have the capability to establish synaptic connections with bipolar cells.

Those skilled in the art will recognize that the versatility of the co-culture approach described herein allows 3DNR and RPE to be combined at different times of photoreceptor maturation in order to produce different three-dimensional tissue products.

### Methods of Making Three Dimensional Tissue Products

Also provided are methods of making a three-dimensional tissue product derived from human induced pluripotent stem cells (hiPSCs) comprising functionally matured retinal pigment epithelial (RPE) cells and a neural retinal patch obtained from three-dimensional neural retina (3DNR), an additional biocompatible component, and a biocompatible scaffold by a) culturing human retinal organoid to generate RPE cells (i.e., cells that are i) obtained from the initial plating or any passage thereafter; ii) at early stages of differentiation; and/or iii) at more advanced stages of differentiation, times in culture, or combinations thereof) and 3DNR; b) separating the RPE cells and the 3DNR; c) seeding the neural retinal patch on top of the RPE cells to form a complex; d) co-culturing the complex in a suitable culture medium and/or e) embedding the neural retinal patch from the 3DNR, the RPE cells or both the neural retinal patch from the 3DNR and the RPE cells in an additional biocompatible component integrated into the product, wherein, following co-culture, the 3DNR, the RPE cells, and the additional biocompatible component physically and functionally integrate to form a three-dimensional tissue product containing a layer of neural retina and an underlying layer of RPE cells, wherein the 3DNR comprises: i) undifferentiated pseudostratified neural retinal epithelium; ii) laminated neural retina tissue including all retinal layers and their corresponding retinal precursor cell types; and/or iii) advanced differentiated retinal tissue including an outer nuclear layer (ONL) and a bipolar cell layer (BCL), wherein the ONL could be rod-enriched, cone-enriched, or any combination thereof, wherein the additional biocompatible component comprises a natural or synthetic compound in a liquid or gel form that provides an appropriate biomechanical environment for cell survival and function, allows manipulation of the product, or both, and wherein the RPE cells are grown on top of the same or a different biocompatible scaffold prior to integration with the 3DNR, and wherein the 3DNR is positioned on top of the RPE cells.

In embodiments, prior to step c), the RPE cells are cultured to generate an RPE monolayer culture (e.g., by i) dissociating RPE cells into a suspension of single RPE cells; ii) plating single RPE cells in an adherent culture (e.g., at a density between about 25,000 and about 300,000 cells per cm² (i.e., approximately 100,000 cells per cm²)); and/or iii) culturing the plated cells in a second culture medium that is not supplemented with exogenous growth factors, morphogenes, or modulators of their signaling pathways, to produce a monolayer of RPE (i.e., a culture medium that supports the growth of the RPE cells). The RPE cells can dissociated into single RPE cells using an enzymatic reaction, an enzyme-free dissociation solution, or mechanical means (i.e., mechanical dissociation).

### Uses of Three Dimensional Tissue Products

Any of the three-dimensional tissue products described herein can be used in a variety of ways. For example, it can be used as a transplant for stem cell-based regenerative therapies for retinal diseases, disorders, or conditions. By way of non-limiting example, these tissue products can be used to treat AMD and/or retinal dystrophies such as retinitis pigmentosa (RP). (See Bharti et al., Invest. Ophthalmolol. Vis Sci 55:1191-1201 (2014); Trounson et al., Cell Stem Cell 17:11-22 (2015)).

Likewise, it can also be used as an in vitro system for studying retinal development, normal mechanisms involving the retina and the RPE, and/or as a disease model for uncovering physiological and/or disease mechanisms and developing therapies. Additionally (or alternatively), the three-dimensional tissue product can also be used as an in vitro model for drug discovery. For example, it can be used to screen for agents that affect retinal development, function, proliferation, maturation, differentiation, and/or survival. These products can also be used to study the toxicology of current treatments.

### Compositions

Provided herein are infusion-ready populations of cells (e.g., RPE cells that have been cultured according to the methods described herein) along with one or more pharmaceutically or veterinarily acceptable carriers, diluents, excipients, or vehicles.

The terms “pharmaceutically acceptable” and “veterinarily acceptable” refer to a pharmaceutically- or veterinarily-acceptable material, composition, or vehicle, such as a liquid or solid filler, diluent, excipient, solvent, or encapsulating material. Each component must be “pharmaceutically acceptable” or “veterinarily acceptable” in the sense of being compatible with the other ingredients of a pharmaceutical formulation. It must also be suitable for use in contact with the tissue or organ of humans and animals without excessive toxicity, irritation, allergic response, immunogenicity, or other problems or complications, commensurate with a reasonable benefit/risk ratio. (See, Remington: The Science and Practice of Pharmacy, 21st Edition; Lippincott Williams & Wilkins: Philadelphia, Pa., 2005; Handbook of Pharmaceutical Excipients, 5th Edition; Rowe et al., Eds., The Pharmaceutical Press and the American Pharmaceutical Association: 2005; and Handbook of Pharmaceutical Additives, 3rd Edition; Ash and Ash Eds., Gower Publishing Company: 2007; Pharmaceutical Preformulation and Formulation, Gibson Ed., CRC Press LLC: Boca Raton, Fla., 2004)).

A pharmaceutical composition of the disclosure is formulated to be compatible with its intended route of administration (i.e., intraocular, subretinal, parenteral, intravenous, intra-arterial, intradermal, subcutaneous, oral, inhalation, transdermal, topical, transmucosal, intraperitoneal or intra-pleural, and/or rectal administration).

Pharmaceutical compositions suitable for injectable use include sterile aqueous solutions (where water soluble) or dispersions and sterile powders for the extemporaneous preparation of sterile injectable solutions or dispersions of cells. In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In some embodiments, it will be desirable to include isotonic agents, for example, sugars, polyalcohols such as manitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound(s) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the disclosure are dictated by and directly dependent on the unique characteristics of the active compound and the particular therapeutic effect to be achieved, and the limitations inherent in the art of compounding such an active compound for the treatment of individuals.

### Kits, Medicaments and Articles of Manufacture

RPE cells cultured according to the methods of the disclosure and/or the three-dimensional tissue products of the disclosure either alone or in combination with one or more other therapeutic agents, may be used in the manufacture of the medicament.

Also provided are kits for treating a retinal disease, disorder, or condition; for examining the role of RPE cells in retinal development; screening for agents that effect retinal development, function, proliferation, maturation, differentiation, and/or survival; and/or examining retinal development, optionally along with instructions for use.

Articles of manufacture are also provided, which include a vessel containing any of the cells or three-dimensional tissue products described herein and instructions for use.

Any of the compositions described herein can be included in a container, pack, or dispenser together with instructions for administration

### Methods of Treatment

Any of the compositions described herein can be used to treat a retinal disease, disorder, or condition in a mammal.

It will be appreciated that administration of therapeutic entities in accordance with the disclosure will be administered with suitable carriers, excipients, and other agents that are incorporated into formulations to provide improved transfer, delivery, tolerance, and the like. A multitude of appropriate formulations can be found in the formulary known to all pharmaceutical chemists: Remington's Pharmaceutical Sciences (15th ed, Mack Publishing Company, Easton, Pa. (1975)), particularly Chapter 87 by Blaug, Seymour, therein. These formulations include, for example, powders, pastes, ointments, jellies, waxes, oils, lipids, lipid (cationic or anionic) containing vesicles (such as Lipofectin™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. Any of the foregoing mixtures may be appropriate in treatments and therapies in accordance with the present disclosure, provided that the active ingredient in the formulation is not inactivated by the formulation and the formulation is physiologically compatible and tolerable with the route of administration. See also Baldrick P. “Pharmaceutical excipient development: the need for preclinical guidance.” Regul. Toxicol Pharmacol. 32(2):210-8 (2000), Wang W. “Lyophilization and development of solid protein pharmaceuticals.” Int. J. Pharm. 203(1-2):1-60 (2000), Charman W N “Lipids, lipophilic drugs, and oral drug delivery-some emerging concepts.” J Pharm Sci. 89(8):967-78 (2000), Powell et al. “Compendium of excipients for parenteral formulations” PDA J Pharm Sci Technol. 52:238-311 (1998) and the citations therein for additional information related to formulations, excipients and carriers well known to pharmaceutical chemists.

The following examples are put forth so as to provide those of ordinary skill in the art with a complete disclosure and description of how the compounds, compositions, articles, devices, and/or methods described and claimed herein are made and evaluated, and are intended to be purely illustrative and are not intended to limit the scope of what the inventors regard as their invention. Efforts have been made to ensure accuracy with respect to numbers (e.g., amounts, temperature, etc.) but some errors and deviations should be accounted for herein. Unless indicated otherwise, parts are parts by weight, temperature is in degrees Celsius or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

### EXAMPLES

### Example 1: Derivation of Retinal Pigment Epithelium from Human Stem Cell-Derived Retinal Organoids

### Methods for Generating the Product:

STEP 1. Generation of Three-Dimensional Retinal Tissue with Functional Photoreceptors from Human iPSCs

Three-dimensional retinal tissue is prepared according to the method described in Zhong et al., Nature Communications 5:4047 (2014) and U.S. Published Patent Application US 2016/0333312, each of which are herein incorporated by reference in its entirety. In such hiPSC-derived 3D retinal organoids, RPE cells are found as a clump at the tip of the retinal organoids.

STEP 2. Isolation of RPE cells and establishment of induced-primary RPE (ipRPE) culture from human 3D retinas (FIG. 1).
1. Dissect RPE tissue from 3D retinal cups and/or floating RPE tissue aggregates and collect them in the center of the petri dish to aspirate the medium.
2. Rinse with PBS (˜5 mL) 2×.
3. After aspirating the last wash, add DMEM media w/0.25% collagenase IV (or other dissociation reagent) and let it sit for 4 hours (weight, mix and warm 15 min; and filter collagenase before use).
4. After 4 hours in incubation (37 C 5% CO2), break the RPE tissue into small pieces by vigorous pipetting.
5. Centrifuge cells at 800 rpm for 5 minutes at 25° C.
6. Aspirate the medium and re-suspend clumps with Accumax (or other dissociation reagent) and allow it to incubate for 20-30 minutes in the incubator.
7. After the allotted time gently pipet clumps until dissociation into single cells, and filter solution with a 40 μm nylon mesh.
8. Cells should be plate 100,000 cell per cm² and grown in RPE medium (Table 4). For example a 12 mm Transwell plated-matrigel coated (or other coating solution) can be used.
9. Change media every 2 days (the first two times rinse with PBS before, just to clean debris and cell death)

TABLE 4
RPE Medium {See Maminishkis et al., Invest.
Ophthalmol Vis Sci. 47(8): 3612-24 (2006)).

| Name | Sigma | Amount | |
|---|---|---|---|
| MEM, α modification | M-4526 | 500 | mL |
| N1 supplement | N-6530 | 5 | mL |
| Glutamine-penicillin-streptomycin | G-1146 | 5 | mL |
| Non essential amino acids | M-7145 | 5 | mL |
| THT* | | | |
| Taurine | T-0625 | 125 | mg |
| Hydrocortisone | H-0396 | 10 | μg |
| Triiodo-thyronin | T-5516 | 0.0065 | μg |

| | |
|---|---|
| Fetal bovine serum, heat inactivated*† | 5% or 15% |

### Example 2: Preparation of Three-Dimensional Tissue Product

hiPSC-derived 3D retinal tissue is generated as described in Zhong et al., Nature Communications 5:4047 (2014) and US Published Patent Application No. 2016/033312. Neural retinal patches (3DNR) are prepared from the 3D retinas using any methods known in the art. For example, hiPSC-derived 3D retinas are opened (e.g., using a tungsten needle or any other method known in the art) in order to expose the inside of the 3D retinal cups, flattened as a retinal flat mount, and retinal explants are obtained. Alternatively, retinal explants can also be obtained directly from 3D retinas using a laser.

These retinal explants (or patches) are then seeded on top of ipRPE (passage 2) and co-cultured for different periods of time. Under these conditions the 3DNR attach to the RPE monolayer forming a 3DNR/RPE complex. (See FIG. 5). Incorporation of an additional biocompatible component (e.g., a natural or synthetic compound in a liquid or gel form that provides an appropriate biomechanical environment for cell survival and function (e.g., a hydrogel) and/or a biocompatible scaffold (e.g., a natural or synthetic scaffold, a scaffold made from biodegradable materials, a scaffold made from non-biodegradable materials, and/or any combinations thereof) into the system further provides an improved biomechanical environment allowing for longer culture periods and manipulation during transplantation. The inclusion of this additional biocompatible component promotes survival and function of the transplanted cells.

### REFERENCES

1. Klimanskaya I, Hipp J, Rezai K A, West M, Atala A, Lanza R. Derivation and comparative assessment of retinal pigment epithelium from human embryonic stem cells using transcriptomics. Cloning Stem Cells. 2004; 6(3):217-45.
2. Buchholz D E, Hikita S T, Rowland T J, Friedrich A M, Hinman C R, Johnson L V, et al. Derivation of functional retinal pigmented epithelium from induced pluripotent stem cells. Stem Cells. 2009; 27(10):2427-34.
3. Zahabi A, Shahbazi E, Ahmadieh H, Hassani S N, Totonchi M, Taei A, et al. A new efficient protocol for directed differentiation of retinal pigmented epithelial cells from normal and retinal disease induced pluripotent stem cells. Stem Cells Dev. 2012; 21(12):2262-72.
4. Buchholz D E, Pennington B O, Croze R H, Hinman C R, Coffey P J, Clegg D O. Rapid and efficient directed differentiation of human pluripotent stem cells into retinal pigmented epithelium. Stem Cells Transl Med. 2013; 2(5):384-93.
5. Maruotti J, Wahlin K, Gorrell D, Bhutto I, Lutty G, Zack D J. A simple and scalable process for the differentiation of retinal pigment epithelium from human pluripotent stem cells. Stem Cells Transl Med. 2013; 2(5):341-54.
6. Singh R, Phillips M J, Kuai D, Meyer J, Martin J M, Smith M A, et al. Functional analysis of serially expanded human iPS cell-derived RPE cultures. Invest Ophthalmol Vis Sci. 2013; 54(10):6767-78.
7. Ferrer M, Corneo B, Davis J, Wan Q, Miyagishima K J, King R, et al. A multiplex high-throughput gene expression assay to simultaneously detect disease and functional markers in induced pluripotent stem cell-derived retinal pigment epithelium. Stem Cells Transl Med. 2014; 3(8):911-22.
8. Reichman S, Terray A, Slembrouck A, Nanteau C, Orieux G, Habeler W, et al. From confluent human iPS cells to self-forming neural retina and retinal pigmented epithelium. P Natl Acad Sci USA. 2014; 111(23):8518-23.
9. Croze R H, Buchholz D E, Radeke M J, Thi W J, Hu Q, Coffey P J, et al. ROCK Inhibition Extends Passage of Pluripotent Stem Cell-Derived Retinal Pigmented Epithelium. Stem Cells Transl Med. 2014; 3(9):1066-78.
10. Leach L L, Buchholz D E, Nadar V P, Lowenstein S E, Clegg D O. Canonical/beta-catenin Wnt pathway activation improves retinal pigmented epithelium derivation from human embryonic stem cells. Invest Ophthalmol Vis Sci. 2015; 56(2):1002-13.
11. Maruotti J, Sripathi S R, Bharti K, Fuller J, Wahlin K J, Ranganathan V, et al. Small-molecule-directed, efficient generation of retinal pigment epithelium from human pluripotent stem cells. P Natl Acad Sci USA. 2015; 112(35):10950-5.
12. Lidgerwood G E, Lim S Y, Crombie D E, Ali R, Gill K P, Hernandez D, et al. Defined Medium Conditions for the Induction and Expansion of Human Pluripotent Stem Cell-Derived Retinal Pigment Epithelium. Stem Cell Rev. 2016; 12(2):179-88.
13. Osakada F, Ikeda H, Mandai M, Wataya T, Watanabe K, Yoshimura N, et al. Toward the generation of rod and cone photoreceptors from mouse, monkey and human embryonic stem cells. Nat Biotechnol. 2008; 26(2):215-24.
14. Vugler A, Carr A J, Lawrence J, Chen L L, Burrell K, Wright A, et al. Elucidating the phenomenon of HESC-derived RPE: anatomy of cell genesis, expansion and retinal transplantation. Exp Neurol. 2008; 214(2):347-61.
15. Idelson M, Alper R, Obolensky A, Ben-Shushan E, Hemo I, Yachimovich-Cohen N, et al. Directed Differentiation of Human Embryonic Stem Cells into Functional Retinal Pigment Epithelium Cells. Cell Stem Cell. 2009; 5(4):396-408.
16. Vaajasaari H, Ilmarinen T, Juuti-Uusitalo K, Rajala K, Onnela N, Narkilahti S, et al. Toward the defined and xeno-free differentiation of functional human pluripotent stem cell-derived retinal pigment epithelial cells. Mol Vis. 2011; 17:558-75.
17. Zhu D, Deng X, Spee C, Sonoda S, Hsieh C L, Barron E, et al. Polarized secretion of PEDF from human embryonic stem cell-derived RPE promotes retinal progenitor cell survival. Invest Ophthalmol Vis Sci. 2011; 52(3):1573-85.
18. Plaza-Reyes A, Petrus-Reurer S, Antonsson L, Stenfelt S, Bartuma H, Panula S, et al. Xeno-Free and Defined Human Embryonic Stem Cell-Derived Retinal Pigment Epithelial Cells Functionally Integrate in a Large-Eyed Preclinical Model. Stem Cell Reports. 2016; 6(1):9-17.
19. Meyer J S, Shearer R L, Capowski E E, Wright L S, Wallace K A, McMillan E L, et al. Modeling early retinal development with human embryonic and induced pluripotent stem cells. Proc Natl Acad Sci USA. 2009; 106(39):16698-703.
20. Zhong X, Gutierrez C, Xue T, Hampton C, Vergara M N, Cao L H, et al. Generation of three-dimensional retinal tissue with functional photoreceptors from human iPSCs. Nat Commun. 2014; 5:4047.
21. Wu W, Zeng Y, Li Z, Li Q, Xu H, Yin Z Q. Features specific to retinal pigment epithelium cells derived from three-dimensional human embryonic stem cell cultures—a new donor for cell therapy. Oncotarget. 2016; 7(16):22819-33.

### EQUIVALENTS

The details of one or more embodiments of the invention are set forth in the accompanying description above. Although any methods and materials similar or equivalent to those described herein can be used in the practice or testing of the present invention, the preferred methods and materials are now described.

The foregoing description has been presented only for the purposes of illustration and is not intended to limit the invention to the precise form disclosed, but by the claims appended hereto.

What is claimed is:

1. A three-dimensional tissue product derived from human induced pluripotent stem cells (hiPSCs) comprising functionally matured retinal pigment epithelial (RPE) cells and a portion of three-dimensional neural retina (3DNR), an additional biocompatible component, and a biocompatible scaffold, wherein the 3DNR, the RPE cells, and the additional biocompatible component are physically and functionally integrated to form a complex containing a layer of neural retina and an underlying layer of RPE cells,
wherein the 3DNR comprises:
i) undifferentiated pseudostratified neural retinal epithelium;
ii) laminated neural retina tissue including all retinal layers and their corresponding retinal precursor cell types; or
iii) advanced differentiated retinal tissue including an outer nuclear layer (ONL) and a bipolar cell layer (BCL), wherein the ONL could be rod-enriched, cone-enriched, or any combination thereof,
wherein the additional biocompatible component comprises a natural or synthetic compound in a liquid or gel form that provides an appropriate biomechanical environment for cell survival and function, allows manipulation of the product, or both, and
wherein the RPE cells are grown on top of said biocompatible scaffold prior to integration with the 3DNR, and wherein the 3DNR is positioned on top of the RPE cells.

2. The three-dimensional tissue product of claim 1, wherein the RPE cells and the 3DNR are both obtained from human retinal organoids.

3. The three-dimensional tissue product of claim 1, wherein the RPE cells are prepared by:
a) culturing human retinal organoids in a first culture medium that is not supplemented with exogenous growth factors, morphogenes, or modulators of their signaling pathways, to generate RPE cells and neural retina (NR);
b) isolating RPE tissue from the cultured retinal organoids;
c) dissociating the isolated RPE tissue into a suspension of single RPE cells;
d) plating single RPE cells in an adherent culture; and
e) culturing the plated cells in a second culture medium that is not supplemented with exogenous growth factors, morphogenes, or modulators of their signaling pathways, to produce a monolayer of RPE.

4. The three-dimensional tissue product of claim 1 or 3, wherein the RPE cells are:
i) obtained from the initial plating or any passage thereafter
ii) at early stages of differentiation; or
iii) at more advanced stages of differentiation, times in culture, or combinations thereof.

5. The three-dimensional tissue product of claim 1, wherein the biocompatible scaffold is selected from the group consisting of natural or synthetic scaffolds, scaffolds made from biodegradable materials, scaffolds made from non-biodegradable materials, or any combinations thereof.

6. A method of making a three-dimensional tissue product derived from human induced pluripotent stem cells (hiPSCs) comprising functionally matured retinal pigment epithelial (RPE) cells and a neural retinal patch obtained from three-dimensional neural retina (3DNR), an additional biocompatible component, and a biocompatible scaffold, the method comprising:
a) culturing human retinal organoid to generate RPE cells and 3DNR;
b) separating the RPE cells and the 3DNR;
c) seeding the neural retinal patch on top of the RPE cells to form a complex;
d) co-culturing the complex in a suitable culture medium; and
e) embedding the neural retinal patch from the 3DNR, the RPE cells or both the neural retinal patch from the 3DNR and the RPE cells in an additional biocompatible component integrated into the product,
wherein, following co-culture, the 3DNR, the RPE cells, and the additional biocompatible component physically and functionally integrate to form a three-dimensional tissue product containing a layer of neural retina and an underlying layer of RPE cells
wherein the 3DNR comprises:
i) undifferentiated pseudostratified neural retinal epithelium;
ii) laminated neural retina tissue including all retinal layers and their corresponding retinal precursor cell types; or
iii) advanced differentiated retinal tissue including an outer nuclear layer (ONL) and a bipolar cell layer (BCL), wherein the ONL could be rod-enriched, cone-enriched, or any combination thereof,
wherein the additional biocompatible component comprises a natural or synthetic compound in a liquid or gel form that provides an appropriate biomechanical environment for cell survival and function, allows manipulation of the product, or both, and
wherein the RPE cells are grown on top of said biocompatible scaffold prior to integration with the 3DNR, and wherein the 3DNR is positioned on top of the RPE cells.

7. The method of claim 6, wherein, prior to step c), the RPE cells are cultured to generate an RPE monolayer culture.

8. The method of claim 7, wherein the RPE monolayer culture is generated by
i) dissociating RPE cells into a suspension of single RPE cells;
ii) plating single RPE cells in an adherent culture; and
iii) culturing the plated cells in a second culture medium that is not supplemented with exogenous growth factors, morphogenes, or modulators of their signaling pathways, to produce a monolayer of RPE.

9. The method of any one of claims 6-8, wherein the RPE cells are:
i) obtained from the initial plating or any passage thereafter;
ii) at early stages of differentiation; or
iii) at more advanced stages of differentiation, times in culture, or combinations thereof.

10. The method of any one of claims 6-9, wherein the RPE cells are dissociated into single RPE cells using an enzymatic reaction, an enzyme-free dissociation solution, or mechanical means.

11. The method of claim 10, wherein the dissociated RPE tissue is mechanically dissociated.

12. The method of claim 10, wherein the single RPE cells are plated at a density between about 25,000 and about 300,000 cells per cm².

13. The method of claim 12, wherein the single RPE cells are plated at a density of approximately 100,000 cells per cm².

14. The method of any one of claims 6-13, wherein the second culture medium supports the growth of the RPE cells.

15. The method of any one of claims 6-14, wherein the biocompatible scaffold is selected from the group consisting of natural or synthetic scaffolds, scaffolds made from biodegradable materials, scaffolds made from non-biodegradable materials, or any combinations thereof.

16. The method of any one of claims 6-15, wherein the 3DNR and the RPE cells are co-cultured at different times of cell maturation.

17. The method of any one of claims 6-15, wherein the 3DNR and the RPE cells are co-cultured in a culture medium that results in a rod-enriched three-dimensional tissue product.

18. The method of any one of claims 6-15, wherein the 3DNR and the RPE cells are co-cultured in a culture medium that results in a cone-enriched three-dimensional tissue product.

19. A method of treating a retinal disease, disorder, or condition, the method comprising transplanting the three-dimensional tissue product of any one of claims 1-5 into an eye of a patient in need thereof.

20. The method of claim 19, wherein the retinal disease, disorder, or condition is selected from the group consisting of retinitis pigmentosa (RP), Leber's congenital amaurosis (LCA), Stargardt disease, Usher's syndrome, choroideremia, a rod-cone or cone-rod dystrophy, a ciliopathy, a mitochondrial disorder, progressive retinal atrophy, a degenerative retinal disease, age related macular degeneration (AMD), wet AMD, dry AMD, geographic atrophy, a familial or acquired maculopathy, a retinal photoreceptor disease, a retinal pigment epithelial-based disease, diabetic retinopathy, cystoid macular edema, uveitis, retinal detachment, traumatic retinal injury, iatrogenic retinal injury, macular holes, macular telangiectasia, a ganglion cell disease, an optic nerve cell disease, glaucoma, optic neuropathy, ischemic retinal disease, retinopathy of prematurity, retinal vascular occlusion, familial macroaneurysm, a retinal vascular disease, an ocular vascular diseases, a vascular disease, and ischemic optic neuropathy.

21. A method of screening for agents that effect retinal development, function, proliferation, maturation, differentiation, survival, or any combination thereof, the method comprising:
a) contacting the three-dimensional tissue product of any one of claims 1-5 with at least one agent; and
b) determining if said agent has an effect on retinal development, function, proliferation, maturation, differentiation, survival, or any combination thereof.

22. The method of claim 21, wherein the at least one agent is a biological agent.

23. The method of claim 22, wherein the biological agent is selected from the group consisting of a growth factor, a trophic factor, a regulatory factor, a hormone, an antibody or an antigen-binding fragment thereof, small molecule, and a peptide.

24. An in vitro method for examining retinal development, the method comprising:
a) preparing the three-dimensional tissue product according to any one of claims 1-5; and
b) monitoring the cellular interaction, function, proliferation, maturation, differentiation, survival, or any combination thereof of cells within the three-dimensional tissue product.

25. The in vitro method of claim 24, wherein the monitoring provides information regarding normal retinal development.

26. The in vitro method of claim 25, wherein the monitoring provides information regarding the interaction of the retina and the RPE.

27. The in vitro method of claim 24, wherein the monitoring provides information regarding retinal abnormal development, diseases, disorders, or conditions.

28. The in vitro method of claim 27, wherein the monitoring provides information regarding underlying mechanisms of retinal abnormal development, diseases, disorders, or conditions.

29. The three-dimensional tissue product of any one of claims 1-5 for use in treating a retinal disease, disorder, or condition, wherein the three-dimensional tissue product of any one of claims 1-5 is for transplantation into an eye of a patient in need thereof.

30. The three-dimensional tissue product for use of claim 29, wherein the retinal disease, disorder, or condition is selected from the group consisting of retinitis pigmentosa (RP), Leber's congenital amaurosis (LCA), Stargardt disease, Usher's syndrome, choroideremia, a rod-cone or cone-rod dystrophy, a ciliopathy, a mitochondrial disorder, progressive retinal atrophy, a degenerative retinal disease, age related macular degeneration (AMD), wet AMD, dry AMD, geographic atrophy, a familial or acquired maculopathy, a retinal photoreceptor disease, a retinal pigment epithelial-based disease, diabetic retinopathy, cystoid macular edema, uveitis, retinal detachment, traumatic retinal injury, iatrogenic retinal injury, macular holes, macular telangiectasia, a ganglion cell disease, an optic nerve cell disease, glaucoma, optic neuropathy, ischemic retinal disease, retinopathy of prematurity, retinal vascular occlusion, familial macroaneurysm, a retinal vascular disease, an ocular vascular diseases, a vascular disease, and ischemic optic neuropathy.
